(19) United States
(12) Patent Application Publication
(10) Pub. No.: US 2025/0332273 A1
(43) Pub. Date: Oct. 30, 2025

(54) SPIDER SILK PROTEIN-BASED HYDROGEL ENABLES INJECTABLE AND SUSTAINED DELIVERY OF PROTEIN THERAPEUTICS FOR NEUROPROTECTION AND AXON REGENERATION

(71) Applicant: The Hong Kong University of Science and Technology, Hong Kong (CN)

(72) Inventors: Fei SUN, Hong Kong (CN); Kai LIU, Hong Kong (CN); Yue LI, Hong Kong (CN); Chao YANG, Hong Kong (CN); Shiyu FANG, Hong Kong (CN)

(21) Appl. No.: 19/187,291

(22) Filed: Apr. 23, 2025

Related U.S. Application Data
(60) Provisional application No. 63/637,897, filed on Apr. 24, 2024.

(51) Int. Cl. A61K 47/64; A61K 47/69; C07K 14/435

(52) U.S. Cl. CPC A61K 47/64; A61K 47/6903; C07K 14/43518

(57) ABSTRACT

The subject invention pertains to a novel injectable protein delivery system and methods for delivering one or more therapeutic agents in the central nervous system (CNS) for promoting axon regeneration. This system is based on the use of a recombinant spider silk protein called spidroin-SpyTag, which undergoes a rapid transition from a sol state to a gel state when exposed to ultrasound treatment and incubated at body temperature. This unique characteristic allows the easy injection of the material into a specific target tissue. The methods herein disclosed allow the delivery of protein therapeutics covalently conjugated to the spidroin-SpyTag to a subject affected by a CNS disorder of injury. Additionally, the method for fabricating this injectable protein delivery system is rapid, convenient, and cost-efficient.

### CROSS-REFERENCE TO RELATED APPLICATION

This application claims the benefit of U.S. Provisional Application Ser. No. 63/637,897, filed Apr. 24, 2024, which is hereby incorporated by reference in its entirety including any tables, figures, or drawings.

### SEQUENCE LISTING

The Sequence Listing for this application is labeled “HK US-203X-SeqList.xml” which was created on Apr. 11, 2025 and is 11,064 bytes. The entire contents of the sequence listing is incorporated herein by reference in its entirety.

### BACKGROUND OF THE INVENTION

Disruption of axon pathways is a common feature of many central nervous system (CNS) disorders, such as spinal cord injury, traumatic brain injury, and neurodegenerative diseases [1-3]. In the adult mammalian CNS, transected axons can hardly regenerate [4, 5]. Currently, no effective treatments exist to stimulate the regeneration of CNS neurons in humans [6]. To promote axon regeneration, researchers are pursuing ways to enhance the intrinsic growth ability of neurons and/or modify extrinsic factors to create an environment conducive to axonal outgrowth [7-9]. Signaling molecules, such as ciliary neurotrophic factor (CNTF) [10, 11], insulin-like growth factor 1 (IGF1) [12, 13], and human neurotrophin-3 (NT-3) [14, 15], have been shown to propel axon regeneration in preclinical studies. However, in contrast to the short half-life in vivo of these signaling molecules, CNS axon regeneration is notoriously reluctant and slow, thus creating a significant impediment to their clinical application. As such, the safe and sustained delivery of these protein drugs poses a significant challenge for materials scientists and engineers. While viral delivery systems, such as adeno-associated viruses, offer a means to deliver functional proteins over an extended period in vivo, their clinical application remains constrained due to concerns regarding cost, complexity, safety, and scalability [16, 17].

Injectable hydrogels, often used in soft tissue engineering, hold promise for addressing the unmet need due to their capacity for precise in situ delivery, minimally invasive administration, and controlled release properties [18-20]. These hydrogel materials are characterized by their high-water content, porous structures, and biocompatibility [21-23]. Various hydrogel systems have been explored for drug release and tissue engineering applications. However, synthetic hydrogels typically necessitate harsh gelation conditions and can generate toxic by-products, while naturally derived polymers like collagen, gelatin, and chitosan often exhibit poor mechanical properties and have the potential to transmit pathogens. Consequently, there is a demand for a hydrogel that can be easily manufacturable, amenable to diverse biofunctionalization, offering non-toxicity, biocompatibility, and favorable mechanical characteristics, and more importantly, capable of rapid sol-gel transition in vivo. These requirements together greatly limit the available options for selecting a suitable material candidate for therapeutic delivery in the nervous system.

Recombinant silk protein hydrogels have emerged as a promising biomaterial that may address these challenges. Natural materials such as silks produced by silkworms and spiders are often the outcomes of the liquid-to-solid phase transition of protein molecules. This unique phase transition behavior, responsive to various stimuli readily available under physiological conditions, opens up the possibility for developing these silk proteins into an injectable therapeutic delivery system. Although previous studies have examined the feasibility of using the Bombyx mori silk protein, fibroin, to create hydrogels for therapeutic delivery and tissue engineering [24-27], the spider silk protein has been underexplored for biomedical applications, likely because of its limited supply from natural sources. In recent years, several versions of recombinant spidroin with good water solubility and high expression yield have been produced using heterologous E. coli expression, making it possible to explore their biomedical applications [28-31]. Compared with those derived from natural fibroin or spidroin, which have been proven to be biocompatible and non-immunogenic [32], but inadequate in biofunctionality, the materials comprising the recombinant proteins are more amenable to biofunctionalization via genetic programming and biomolecular engineering. Although several studies have demonstrated the benefits of natural spidroin-based materials for peripheral nerve repair, their efficacy in the CNS remains unproven. Given these limitations, there is an urgent need to develop alternative strategies for noninvasive, safe, and sustained delivery of proteins in CNS.

### BRIEF SUMMARY OF THE INVENTION

The subject invention addresses the need for an effective and minimally invasive protein delivery system for promoting axon regeneration in the CNS. In a first aspect, the subject invention discloses a novel injectable protein delivery system comprising a material based on the use of a recombinant spider silk protein called spidroin-SpyTag. In preferred embodiments, spidroin-SpyTag can be injected into a target tissue. The spidroin-SpyTag undergoes a rapid sol-gel transition when exposed to brief sonication and at a temperature of about 37° C., enabling its injectability.

In certain embodiments, the application of SpyTag/SpyCatcher click chemistry allows the functionalization of spidroin-SpyTag with various bioactive motifs, including cell-binding ligands and neurotrophic factors. This versatility makes it suitable for neuronal culturing and for tailored therapeutic interventions. In certain embodiments, spidroin-SpyTag is in hydrogel injectable form and comprises one or more bioactive agents covalently conjugated to the spidroin-SpyTag. In certain embodiments, the one or more bioactive agents are protein therapeutics comprising one or more of ciliary neurotrophic factor (CNTF), insulin-like growth factor (IGF1), laminin, or osteopontin (OPN).

In a second aspect, the subject invention discloses a method for delivering the one or more therapeutic agents to a targeted CNS tissue, comprising administering the spidroin-SpyTag hydrogel covalently conjugated to protein therapeutics to the targeted CNS tissue of a subject in need thereof. In certain embodiments, the targeted CNS tissue comprises a site affected by a CNS disorder or injury. In certain embodiments, the CNS disorder or injury comprises spinal cord injury, traumatic brain injury, stroke, glaucoma, muscle dystrophy, muscle hypertrophy, metabolic myopathies, or muscle paralysis. In certain embodiments, the site of injury comprises an optic nerve and a retinal tissue. In certain embodiments, the spidroin-SpyTag protein delivery system provides an injectable and sustained delivery of proteins for promoting neuroprotection and neuroregeneration of a CNS tissue affected by a disorder or injury.

### BRIEF DESCRIPTION OF THE DRAWINGS

FIG. 1 provides a schematic showing the creation and applications of recombinant Spidroin-SpyTag (S-A) hydrogel. Pretreatment with ultrasound accelerates the thermally induced sol-gel transition of S-A. Through SpyTag/SpyCatcher chemistry, S-A hydrogel covalently immobilizes a variety of SpyCatcher-fusion proteins of interest (i.e., B-POI-B), such as B-CNTF-B. The resulting hydrogels are applicable for neuronal culture and optic nerve regeneration.

FIGS. 2A-2H illustrate how ultrasound facilitates the thermally induced sol-gel transition of S-A. Evolution of G′ and G″ of S-A (4 wt %) at 37° C. as a function of time (FIG. 2A). Evolution of G′ and G″ of sonicated S-A (4 wt %) at 37° C. as a function of time (FIG. 2B). Frequency-sweep test of S-A hydrogel (4 wt %) with the strain fixed at 5% (FIG. 2C). Strain-sweep test of S-A hydrogel (4 wt %) with the frequency fixed at 1 rad/s (FIG. 2D). Erosion profiles of S-A hydrogel (4 wt %, 30 μl) immersed in 0.5 ml of Tris buffer, PBS, or Milli-Q® water (FIG. 2E). Data are presented as mean±SD (n=3). CD spectra of S-A before and after sonication (FIG. 2F). TEM images of the S-A solution before sonication, the S-A solution after sonication (i.e., pre-gel solution), and the resuspended S-A hydrogel (FIG. 2G). Scale bars, 100 nm. Photograph of S-A hydrogel (4 wt %). Scale bars, 5 mm (FIG. 2H).

FIGS. 3A-3C illustrate the biofunctionalization of S-A hydrogels via SpyTag/SpyCatcher chemistry. Spontaneous isopeptide bond-forming SpyTag/SpyCatcher reaction (FIG. 3A). SDS-PAGE analysis of the reactions of S-A and SpyCatcher-fusion proteins (FIG. 3B). Comparison of release profiles of proteins that are physically and covalently encapsulated within S-A gels (FIG. 3C). Data are presented as mean±SD (n=3).

FIGS. 4A-4C illustrate culturing of N2A cells on S-A hydrogels decorated with functional proteins. FIG. 4A shows representative images of N2A cells grown on hydrogels for 3 days. Scale bar, 50 μm. FIG. 4B illustrates cell viability percentages on the blank S-A gel, B-CNTF-B gel, and B-LM-B gel were determined after 3 days of culturing. One-way ANOVA followed by Tukey's multiple comparisons test was performed, *P≤0.05. Data are presented as mean±SD (n=5). FIG. 4C illustrates cell densities on the three types of hydrogels, with normalized data shown as mean±SD (n=5).

FIGS. 5A-5D show growth of dorsal root ganglion (DRG) neurons on blank or functionalized S-A hydrogels. FIG. 5A shows the influence of immobilized factors on the adhesion of DRG neurons. Cells were stained by Tuj1 antibody after culturing for 16 hours. Scale bars, 100 μm. FIG. 5B illustrates cell densities on different substrates. One-way ANOVA followed by Dunnet's multiple comparisons test was performed. Data are presented as mean±SEM (n=3). FIG. 5C shows the influence of immobilized neurotrophic factors on neurite growth. Representative images of neurons after Tuj1 staining are shown. Scale bars, 25 μm.

FIG. 5D illustrates the average neurite length per neuron on different substrates. One-way ANOVA followed by Dunnet's multiple comparisons test was performed. Data are presented as mean±SEM (n=3). ns indicates no significant difference. *P≤0.05; **P≤0.01. *, **, or ns above a line denotes comparisons between the two groups connected by that line, while *, **, or ns elsewhere refers to comparisons with the PDL/Laminin group by default.

FIG. 6 illustrates degradation of S-A hydrogels after injection into the vitreous body. Retinal sections were collected at various time points (1, 5, 14, and 28 days) following intravitreal injection of vehicle control, S-A gel, B-CNTF-B solution (15 μM), or B-CNTF-B gel (15 μM) immediately after optic nerve crush. According to the histological analysis, the gels remained after two weeks but had disappeared by four weeks. White arrows indicate the presence of the gels.

FIGS. 7A-7D illustrate immunogenic effects of the S-A hydrogels. Retinal sections were collected at various time points (1, 5, 14, and 28 days) following intravitreal injection of vehicle control or S-A gel immediately after optic nerve crush. The samples were stained with Iba1 (FIG. 7A), CD4 (FIG. 7B), CD68 (FIG. 7C), or CD45 (FIG. 7D) antibodies and DA PI (blue). Representative images from retinas at 5 days post-crush are shown. Scale bars, 50 μm. Bar graphs show the densities of microglia (Iba1-positive) (FIG. 7A), helper T cells (CD4-20 positive) (FIG. 7B), activated macrophages and microglia (CD68-positive) (FIG. 7C), or hematopoietic cells (CD45-positive) (FIG. 7D) in the retina. ns, not significant. *P≤0.05. Multiple t tests were performed. Data are presented as mean±SEM (n=3-4 mice).

FIGS. 8A-8B illustrate B-CNTF-B S-A hydrogel enabling prolonged activation of STAT3 signaling in vivo. Retinal sections from C57B L/6 mice were injected with vehicle, blank gel, B-CNTF-B solution (15 μM), or B-CNTF-B gel (15 μM) immediately after optic nerve crush and were collected at different time points post-injection (1, 5, 14, or 28 days) (FIG. 8A). The samples were stained with Tuj1 (green) and p-STAT3 (Tyr705) (magenta) antibodies. Scale bars, 20 μm. Quantification of p-STAT3-positive RGCs (FIG. 8B). ns, not significant. **P≤0.01. One-way ANOVA followed by Bonferroni or Games-Howell multiple comparisons test was performed. Data are presented as mean±SEM (n=4-6 mice).

FIGS. 9A-9D illustrate B-CNTF-B hydrogel enhancing RGC survival. Whole-mount retinas from C57BL/6 mice were injected with vehicle, blank S-A gel, B-CNTF-B solution (15 M), or B-CNTF-B gel (15 μM) immediately after optic nerve crush and were collected 14 days (FIG. 9A) or 28 days (FIG. 9C) post-crush. RGCs were labeled by Tuj1 antibody (green). Scale bar: 20 μm. Quantification of live RGCs at 14 days (FIG. 9B) or 28 days (FIG. 9D) post-crush. ns, not significant. *P≤0.05. **P≤0.01. One-way ANOVA followed by Bonferroni multiple comparisons test was conducted. Data are presented as means±SEM (n=5-6 mice).

FIGS. 10A-10D illustrate B-CNTF-B hydrogel promoting axon regeneration in optic nerve. Optic nerve sections from C57BL/6 mice were injected with vehicle, blank gel, B-CNTF-B solution (15 μM), or B-CNTF-B gel (15 μM) immediately after optic nerve crush and were collected 14 days post-crush (FIG. 10A) and 28 days post-crush (FIG. 10C). Axons were labeled by CTB-FITC intravitreal injection 2 days before sacrificing the animals. The samples were stained with FITC (red) antibody. Scale bars, 200 μm. Quantification of the regenerating axons that crossed indicated distances from the lesion sites at 14 days post-crush (FIG. 10B) and 28 days post-crush (FIG. 10D). ns, not significant. *P≤0.05. **P≤0.01. One-way ANOVA followed by Bonferroni or Games-Howell multiple comparisons test was performed. Data are presented as mean±SEM (n=5-6 mice).

FIGS. 11A-11F illustrate that covalent immobilization is essential for the functional duration of CNTF in vivo. FIG. 11A shows retinal sections from C57B L/6 mice administered either physically mixed CNTF gel (S-A+15 μM CNTF) or covalently bound B-CNTF-B gel (S-A+15 UM B-CNTF-B) via intravitreal injection immediately after optic nerve crush, collected at different time points post-crush (14 and 28 days). The samples were stained with Tuj1 (green) and p-STAT3 (magenta) antibodies. Scale bar: 20 μm. FIG. 11B illustrates the quantification of p-STAT 3-positive RGCs at various time points (1, 5, 14, or 28 days). ns, not significant; **P≤0.01. Two-way ANOVA followed by Bonferroni multiple comparisons test was performed. Data are presented as mean±SEM (n=3 mice). FIG. 11C shows whole-mount retinas from C57BL/6 mice administered with either physically mixed CNTF gels or covalently bound B-CNTF-B gel via intravitreal injection immediately after optic nerve crush, collected 14 days post-crush. RGCs were labeled with Tuj1 antibody (green). Scale bar: 20 μm. FIG. 11D illustrates the percentage of surviving RGCs at 14 days and 28 days post-crush. *P≤0.05; **P≤0.01. An unpaired t-test was conducted. Data are presented as means±SEM (n=3 mice). FIG. 11E shows optic nerve sections from mice injected with either physically mixed CNTF gels or covalently bound B-CNTF-B gel after optic nerve crush. The samples were collected 14 days post-crush, and stained with FITC (red) antibody to detect CTB-FITC traced axons. Scale bar: 200 μm. FIG. 11F illustrates the quantification of regenerating axons that crossed indicated distances from the lesion sites at 14 days post-crush and 28 days post-crush. **P≤0.01. Two-way ANOVA followed by Bonferroni multiple comparisons test was performed. Data are presented as mean±SEM (n=3 mice).

TABLE 1
Gene sequences used in this study.
Gene sequence of SpyTag (SEQ ID NO: 1):

| | |
|---|---|
| 1 | GCACATATTG TTATGGTTGA CGCTTATAAG CCAACAAAA |

Gene sequence of SpyCatcher (SEQ ID NO: 2):

| | |
|---|---|
| 1 | GCCATGGTTG ATACCTTATC AGGTTTATCA AGTGAGCAAG |
| 41 | GTCAGTCCGG TGATATGACA ATTGAAGAAG ATAGTGCTAC |
| 81 | CCATATTAAA TTCTCAAAAC GTGATGAGGA CGGCAAAGAG |
| 121 | TTAGCTGGTG CAACTATGGA GTTGCGTGAT TCATCTGGTA |
| 161 | AAACTATTAG TACATGGATT TCAGATGGAC AAGTGAAAGA |
| 201 | TTTCTACCTG TATCCAGGAA AATATACATT TGTCGAAACC |
| 241 | GCAGCACCAG ACGGTTATGA GGTAGCAACT GCTATTACCT |
| 281 | TTACAGTTAA TGAGCAAGGT CAGGTTACTG TAAATGGCAA |
| 321 | AGCAACTAAA GGTGACGCTC ATATTGAC |

TABLE 2
Amino acid sequences of proteins
used in this study.
Amino acid sequence of SpyTag (SEQ ID NO: 3):

| | |
|---|---|
| 1 | AHIVMVDAYK PTK |

Amino acid sequence of NTD2RepCTD-SpyTag (S-A)
(SEQ ID NO: 4):

| | |
|---|---|
| 1 | HMSHTTPWTN PGLAENFMNS FMQGLSSMPG FTASQLDDMS |
| 41 | TIAQSMVQSI QSLAAQGRTS PNKLQALNMA FASSMAEIAA |
| 81 | SEEGGGSLST KTSSIASAMS NAFLQTTGVV NQPFINEITQ |
| 121 | LVSMFAQAGM NDVSAGNSGR GQGGYGQGSG GNAAAAAAAA |
| 161 | AAAAAAAGQG GQGGYGRQSQ GAGSAAAAAA AAAAAAAAGS |
| 201 | GQGGYGGQGQ GGYGQSGNSV TSGGYGYGTS AAAGAGVAAG |
| 241 | SYAGAVNRLS SAEAASRVSS NIAAIASGGA SALPSVISNI |
| 281 | YSGVVASGVS SNEALIQALL ELLSALVHVL SSASIGNVSS |
| 321 | VGVDSTLNVV QDSVGQYVGG TGGGGSGGGG SGGGGSASAH |
| 361 | IVMVDAYKPT KLEHHHHHH |

Note:
The sequence underlined is NTD (M aSp1,
Euprosthenops australis, EM BL accession
number A M 259067);
The sequence in italics is 2R ep (M aSp1,
Euprosthenops australis, EM BL accession
number AJ 973155);
The sequence in bold is CTD (MiSp, A raneus
ventricosus, GenBank accession number
JX 513956);
GGGGSGGGG SGGGGS (residues 342-351 of SEQ ID
NO: 4) is a flexible linker.
Amino acid sequence of SpyCatcher-ELP-LM-ELP-
SpyCatcher (B-LM-B) (SEQ ID NO: 5):

| | |
|---|---|
| 1 | MKGSSHHHHH HVDIPTTENL YFQGAMVDTL SGLSSEQGQS |
| 41 | GDMTIEEDSA THIKFSKRDE DGKELAGATM ELRDSSGKTI |
| 81 | STWISDGQVK DFYLYPGKYT FVETAAPDGY EVATAITFTV |
| 121 | NEQGQVTVNG KATKGDAHID GPQGIWGQLE GHGVGVPGVG |
| 161 | VPGVGVPGEG VPGVGVPGVG VPGVGVPGVG VPGEGVPGVG |
| 201 | VPGVGVPGVG VPGVGVPGEG VPGVGVPGVG ELTFALRGDN |
| 241 | PVPMSMRGGK LTWQELYQLK YKGITSVPGV GVPGVGVPGE |
| 281 | GVPGVGVPGV GVPGVGVPGV GVPGEGVPGV GVPGVGVPGV |
| 321 | GVPGVGVPGE GVPGVGVPGV GVPGGLVDIP TTENLYFQGA |
| 361 | MVDTLSGLSS EQGQSGDMTI EEDSATHIKF SKRDEDGKEL |
| 401 | AGATMELRDS SGKTISTWIS DGQVKDFYLY PGKYTFVETA |
| 441 | APDGYEVATA ITFTVNEQGQ VTVNGKATKG DAHIDGPQGI |
| 481 | WGQLEWKK |

Amino acid sequence of SpyCatcher-ELP-CNTF-ELP-
SpyCatcher (B-CNTF-B) (SEQ ID NO: 6):

| | |
|---|---|
| 1 | MKGSSHHHHH HVDIPTTENL YFQGAMVDTL SGLSSEQGQS |
| 41 | GDMTIEEDSA THIKFSKRDE DGKELAGATM ELRDSSGKTI |
| 81 | STWISDGQVK DFYLYPGKYT FVETAAPDGY EVATAITFTV |
| 121 | NEQGQVTVNG KATKGDAHID GPQGIWGQLE GHGVGVPGVG |
| 161 | VPGVGVPGEG VPGVGVPGVG VPGVGVPGVG VPGEGVPGVG |
| 201 | VPGVGVPGVG VPGVGVPGEG VPGVGVPGVG ELAFAEQTPL |
| 241 | TLHRRDLCSR SIWLARKIRS DLTALMESYV KHQGLNKNIN |
| 281 | LDSVDGVPVA STDRWSEMTE AERLQENLQA YRTFQGMLTK |
| 321 | LLEDQRVHFT PTEGDFHQAI HTLMLQVSAF AYQLEELMVL |
| 361 | LEQKIPENEA DGMPATVGDG GLFEKKLWGL KVLQELSQWT |
| 401 | VRSIHDLRVI SSHQMGISAL ESHYGAKDKQ MTSVPGVGVP |
| 441 | GVGVPGEGVP GVGVPGVGVP GVGVPGVGVP GEGVPGVGVP |
| 481 | GVGVPGVGVP GVGVPGEGVP GVGVPGVGVP GGLVDIPTTE |
| 521 | NLYFQGAMVD TLSGLSSEQG QSGDMTIEED SATHIKFSKR |
| 561 | DEDGKELAGA TMELRDSSGK TISTWISDGQ VKDFYLYPGK |
| 601 | YTFVETAAPD GYEVATAITF TVNEQGQVTV NGKATKGDAH |
| 641 | IDGPQGIWGQ LEWKK |

Amino acid sequence of SpyCatcher-ELP-
Insulin-like growth factor 1-ELP-
SpyCatcher (B-IGF1-B) (SEQ ID NO: 7):

| | |
|---|---|
| 1 | MKGSSHHHHH HVDIPTTENL YFQGAMVDTL SGLSSEQGQS |
| 41 | GDMTIEEDSA THIKFSKRDE DGKELAGATM ELRDSSGKTI |
| 81 | STWISDGQVK DFYLYPGKYT FVETAAPDGY EVATAITFTV |
| 121 | NEQGQVTVNG KATKGDAHID GPQGIWGQLE GHGVGVPGVG |
| 161 | VPGVGVPGEG VPGVGVPGVG VPGVGVPGVG VPGEGVPGVG |
| 201 | VPGVGVPGVG VPGVGVPGEG VPGVGVPGVG ELGPETLCGA |
| 241 | ELVDALQFVC GDRGFYFNKP TGYGSSSRRA PQTGIVDECC |
| 281 | FRSCDLRRLE MYCAPLKPAK SATSVPGVGV PGVGVPGEGV |
| 321 | PGVGVPGVGV PGVGVPGVGV PGEGVPGVGV PGVGVPGVGV |
| 361 | PGVGVPGEGV PGVGVPGVGV PGGLVDIPTT ENLYFQGAMV |
| 401 | DTLSGLSSEQ GQSGDMTIEE DSATHIKFSK RDEDGKELAG |
| 441 | ATMELRDSSG KTISTWISDG QVKDFYLYPG KYTFVETAAP |
| 481 | DGYEVATAIT FTVNEQGQVT VNGKATKGDA HIDGPQGIWG |
| 521 | QLEWKK |

Amino acid sequence of SpyCatcher-ELP-
Osteopontin -ELP-SpyCatcher (B-OPN-B)
(SEQ ID NO: 8):

| | |
|---|---|
| 1 | MKGSSHHHHH HVDIPTTENL YFQGAMVDTL SGLSSEQGQS |
| 41 | GDMTIEEDSA THIKFSKRDE DGKELAGATM ELRDSSGKTI |
| 81 | STWISDGQVK DFYLYPGKYT FVETAAPDGY EVATAITFTV |
| 121 | NEQGQVTVNG KATKGDAHID GPQGIWGQLE GHGVGVPGVG |
| 161 | VPGVGVPGEG VPGVGVPGVG VPGVGVPGVG VPGEGVPGVG |
| 201 | VPGVGVPGVG VPGVGVPGEG VPGVGVPGVG ELIPVKQADS |
| 241 | GSSEEKQLYN KYPDAVATWL NPDPSQKQNL LAPQNAVSSE |
| 281 | ETNDFKQETL PSKSNESHDH MDDMDDEDDD DHVDSQDSID |
| 321 | SNDSDDVDDT DDSHQSDESH HSDESDELVT DFPTDLPATE |
| 361 | VFTPVVPTVD TYDGRGDSVV YGLRSKSKKF RRPDIQYPDA |
| 401 | TDEDITSHME SEELNGAYKA IPVAQDLNAP SDWDSRGKDS |
| 441 | YETSQLDDQS AETHSHKQSR LYKRKANDES NEHSDVIDSQ |
| 481 | ELSKVSREFH SHEFHSHEDM LVVDPKSKEE DKHLKFRISH |
| 521 | ELDSASSEVN TSVPGVGVPG VGVPGEGVPG VGVPGVGVPG |
| 561 | VGVPGVGVPG EGVPGVGVPG VGVPGVGVPG VGVPGEGVPG |
| 601 | VGVPGVGVPG GLVDIPTTEN LYFQGAMVDT LSGLSSEQGQ |
| 641 | SGDMTIEEDS ATHIKFSKRD EDGKELAGAT MELRDSSGKT |
| 681 | ISTWISDGQV KDFYLYPGKY TFVETAAPDG YEVATAITFT |
| 721 | VNEQGQVTVN GKATKGDAHI DGPQGIWGQL EWKK |

### BRIEF DESCRIPTION OF THE SEQUENCES

SEQ ID NO: 1:
5′-GCACATATTGTTATGGTTGACGCTTATAAGCCAACAAAA-3′
Gene sequence of SpyTag
SEQ ID NO: 2:
5′-GCCATGGTTGATACCTTATCAGGTTTATCAAGTGAGCAAGGTCAGTC
CGGTGATATGACAATTGAAGAAGATAGTGCTACCCATATTAAATTCTCAA
AACGTGATGAGGACGGCAAAGAGTTAGCTGGTGCAACTATGGAGTTGCGT
GATTCATCTGGTAAAACTATTAGTACATGGATTTCAGATGGACAAGTGAA
AGATTTCTACCTGTATCCAGGAAAATATACATTTGTCGAAACCGCAGCAC
CAGACGGTTATGAGGTAGCAACTGCTATTACCTTTACAGTTAATGAGCAA
GGTCAGGTTACTGTAAATGGCAAAGCAACTAAAGGTGACGCTCATATTGA
C-3′
Gene sequence of SpyCatcher
SEQ ID NO: 3:
AHIVMVDAYKPTK
Amino acid sequence of SpyTag:
SEQ ID NO: 4:
HMSHTTPWTNPGLAENFMNSFMQGLSSMPGFTASQLDDMSTIAQSMVQSI
QSLAAQGRTSPNKLQALNMAFASSMAEIAASEEGGGSLSTKTSSIASAMS
NAFLQTTGVVNQPFINEITQLVSMFAQAGMNDVSAGNSGRGQGGYGQGSG
GNAAAAAAAAAAAAAAAGQGGQGGYGRQSQGAGSAAAAAAAAAAAAAAGS
GQGGYGGQGQGGYGQSGNSVTSGGYGYGTSAAAGAGVAAGSYAGAVNRLS
SAEAASRVSSNIAAIASGGASALPSVISNIYSGVVASGVSSNEALIQALL
ELLSALVHVLSSASIGNVSSVGVDSTLNVVQDSVGQYVGGTGGGGSGGGG
SGGGGSASAHIVMVDAYKPTKLEHHHHHH
Amino acid sequence of NTD 2RepCTD-
SpyTag (S-A)
SEQ ID NO: 5:
MKGSSHHHHHHVDIPTTENLYFQGAMVDTLSGLSSEQGQSGDMTIEEDSA
THIKFSKRDEDGKELAGATMELRDSSGKTISTWISDGQVKDFYLYPGKYT
FVETAAPDGYEVATAITFTVNEQGQVTVNGKATKGDAHIDGPQGIWGQLE
GHGVGVPGVGVPGVGVPGEGVPGVGVPGVGVPGVGVPGVGVPGEGVPGVG
VPGVGVPGVGVPGVGVPGEGVPGVGVPGVGELTFALRGDNPVPMSMRGGK
LTWQELYQLKYKGITSVPGVGVPGVGVPGEGVPGVGVPGVGVPGVGVPGV
GVPGEGVPGVGVPGVGVPGVGVPGVGVPGEGVPGVGVPGVGVPGGLVDIP
TTENLYFQGAMVDTLSGLSSEQGQSGDMTIEEDSATHIKFSKRDEDGKEL
AGATMELRDSSGKTISTWISDGQVKDFYLYPGKYTFVETAAPDGYEVATA
ITFTVNEQGQVTVNGKATKGDAHIDGPQGIWGQLEWKK
Amino acid sequence of SpyCatcher-ELP-
LM-ELP-SpyCatcher (B-LM-B)
SEQ ID NO: 6:
MKGSSHHHHHHVDIPTTENLYFQGAMVDTLSGLSSEQGQSGDMTIEEDSA
THIKFSKRDEDGKELAGATMELRDSSGKTISTWISDGQVKDFYLYPGKYT
FVETAAPDGYEVATAITFTVNEQGQVTVNGKATKGDAHIDGPQGIWGQLE
GHGVGVPGVGVPGVGVPGEGVPGVGVPGVGVPGVGVPGVGVPGEGVPGVG
VPGVGVPGVGVPGVGVPGEGVPGVGVPGVGELAFAEQTPLTLHRRDLCSR
SIWLARKIRSDLTALMESYVKHQGLNKNINLDSVDGVPVASTDRWSEMTE
AERLQENLQAYRTFQGMLTKLLEDQRVHFTPTEGDFHQAIHTLMLQVSAF
AYQLEELMVLLEQKIPENEADGMPATVGDGGLFEKKLWGLKVLQELSQWT
VRSIHDLRVISSHQMGISALESHYGAKDKQMTSVPGVGVPGVGVPGEGVP
GVGVPGVGVPGVGVPGVGVPGEGVPGVGVPGVGVPGVGVPGVGVPGEGVP
GVGVPGVGVPGGLVDIPTTENLYFQGAMVDTLSGLSSEQGQSGDMTIEED
SATHIKFSKRDEDGKELAGATMELRDSSGKTISTWISDGQVKDFYLYPGK
YTFVETAAPDGYEVATAITFTVNEQGQVTVNGKATKGDAHIDGPQGIWGQ
LEWKK
Amino acid sequence of SpyCatcher-ELP-
CNTF-ELP-SpyCatcher (B-CNTF-B)
SEQ ID NO: 7:
MKGSSHHHHHHVDIPTTENLYFQGAMVDTLSGLSSEQGQSGDMTIEEDSA
THIKFSKRDEDGKELAGATMELRDSSGKTISTWISDGQVKDFYLYPGKYT
FVETAAPDGYEVATAITFTVNEQGQVTVNGKATKGDAHIDGPQGIWGQLE
GHGVGVPGVGVPGVGVPGEGVPGVGVPGVGVPGVGVPGVGVPGEGVPGVG
VPGVGVPGVGVPGVGVPGEGVPGVGVPGVGELGPETLCGAELVDALQFVC
GDRGFYFNKPTGYGSSSRRAPQTGIVDECCFRSCDLRRLEMYCAPLKPAK
SATSVPGVGVPGVGVPGEGVPGVGVPGVGVPGVGVPGVGVPGEGVPGVGV
PGVGVPGVGVPGVGVPGEGVPGVGVPGVGVPGGLVDIPTTENLYFQGAMV
DTLSGLSSEQGQSGDMTIEEDSATHIKFSKRDEDGKELAGATMELRDSSG
KTISTWISDGQVKDFYLYPGKYTFVETAAPDGYEVATAITFTVNEQGQVT
VNGKATKGDAHIDGPQGIWGQLEWKK
Amino acid sequence of SpyCatcher-ELP-
Insulin-like growth factor 1-EL P-
SpyCatcher (B-IGF1-B)
SEQ ID NO: 8:
MKGSSHHHHHHVDIPTTENLYFQGAMVDTLSGLSSEQGQSGDMTIEEDSA
THIKFSKRD+NLEDGKELAGATMELRDSSGKTISTWISDGQVKDFYLYPG
KYTFVETAAPDGYEVATAITFTVNEQGQVTVNGKATKGDAHIDGPQGIWG
QLEGHGVGVPGVGVPGVGVPGEGVPGVGVPGVGVPGVGVPGVGVPGEGVP
GVGVPGVGVPGVGVPGVGVPGEGVPGVGVPGVGELIPVKQADSGSSEEKQ
LYNKYPDAVATWLNPDPSQKQNLLAPQNAVSSEETNDFKQETLPSKSNES
HDHMDDMDDEDDDDHVDSQDSIDSNDSDDVDDTDDSHQSDESHHSDESDE
LVTDFPTDLPATEVFTPVVPTVDTYDGRGDSVVYGLRSKSKKFRRPDIQY
PDATDEDITSHMESEELNGAYKAIPVAQDLNAPSDWDSRGKDSYETSQLD
DQSAETHSHKQSRLYKRKANDESNEHSDVIDSQELSKVSREFHSHEFHSH
EDMLVVDPKSKEEDKHLKFRISHELDSASSEVNTSVPGVGVPGVGVPGEG
VPGVGVPGVGVPGVGVPGVGVPGEGVPGVGVPGVGVPGVGVPGVGVPGEG
VPGVGVPGVGVPGGLVDIPTTENLYFQGAMVDTLSGLSSEQGQSGDMTIE
EDSATHIKFSKRDEDGKELAGATMELRDSSGKTISTWISDGQVKDFYLYP
GKYTFVETAAPDGYEVATAITFTVNEQGQVTVNGKATKGDAHIDGPQGIW
GQLEWKK
Amino acid sequence of SpyCatcher-ELP-
Osteopontin-ELP-SpyCatcher (B-OPN-B)

### DETAILED DISCLOSURE OF THE INVENTION

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the terms “about” are used, these compositions contain the stated amount of the ingredient with a variation (error range) of 0-10% around the value (X±10%). In other contexts, the term “about” is providing a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

As used herein, the terms “therapeutically-effective amount,” “therapeutically-effective dose,” “effective amount,” and “effective dose” are used to refer to an amount or dose of a compound or composition that, when administered to a subject, is capable of treating, preventing, or improving a condition, disease, or disorder in a subject. In other words, when administered to a subject, the amount is “therapeutically effective.” The actual amount will vary depending on a number of factors including, but not limited to, the particular condition, disease, or disorder being treated, prevented, or improved; the severity of the condition; the weight, height, age, and health of the patient; and the route of administration.

As used herein, the term “treatment” refers to eradicating; reducing; ameliorating; abatement; remission; diminishing of symptoms or delaying the onset of symptoms; slowing in the rate of degeneration or decline; making the final point of degeneration less debilitating; and/or improving a subject's physical or mental well-being or reversing a sign or symptom of a health condition, disease or disorder to any extent, and includes, but does not require, a complete cure of the condition, disease, or disorder. Treating can be curing, improving, or partially ameliorating a disorder. “Treatment” can also include improving or enhancing a condition or characteristic, for example, bringing the function of a particular system in the body to a heightened state of health or homeostasis.

As used herein, “subject” refers to an animal, such as a mammal, for example a human. The methods described herein can be useful in both humans and non-human animals. In some embodiments, the subject is a mammal (such as an animal model of disease), and in some embodiments, the subject is a human. The terms “subject” and “patient” can be used interchangeably. The animal may be for example, humans, pigs, horses, goats, cats, mice, rats, dogs, apes, fish, chimpanzees, orangutans, guinea pigs, hamsters, cows, sheep, birds, chickens, as well as any other vertebrate or invertebrate. The preferred subject in the context of this invention is a human. The subject can be of any age or stage of development, including infant, toddler, adolescent, teenager, adult, or senior.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

These benefits can include, but are not limited to, the treatment of a health condition, disease, or disorder; prevention of a health condition, disease or disorder; immune health; enhancement of the function of an organ, tissue, or system in the body.

### Injectable Protein Delivery System and Methods for Delivering a Therapeutic Agent to a CNS Targeted Tissue

The subject invention discloses a novel injectable protein delivery system for promoting neuroprotection and axon regeneration in the central nervous system (CNS). In a first aspect, the subject invention comprises an injectable protein delivery system comprising a recombinant spider silk protein called spidroin-SpyTag. In certain embodiments, spidroin-SpyTag undergoes a rapid transition from a sol state to a gel state when exposed to brief ultrasound treatment and incubated at body temperature. In preferred embodiments, spidroin-SpyTag transitions from a sol state to a gel state after brief sonication and incubation at a temperature of about 37° C. This unique characteristic allows for the easy injection of the material into specific targeted tissues. In certain embodiments, spidroin-SpyTag is conjugated to one or more bioactive agents or protein therapeutics, including, but not limited to, ciliary neurotrophic factor (CNTF), insulin-like growth factor (IGF1), laminin, or osteopontin (OPN). In preferred embodiments, the subject invention comprises spidroin-SpyTag in hydrogel form conjugated with a bioactive agent or protein therapeutic.

In a second aspect, the subject invention discloses methods for delivering a bioactive agent or protein therapeutic to a CNS target comprising administering the spidroin-SpyTag in hydrogel form conjugated to the bioactive agent or protein therapeutic to a targeted CNS tissue of a subject in need thereof. In certain embodiments, a therapeutically effective amount of spidroin-SpyTag in hydrogel form covalently conjugated with one or more protein therapeutics, including, but not limited to, ciliary neurotrophic factor (CNTF), insulin-like growth factor (IGF1), laminin, or osteopontin (OPN) is administered to the subject. In certain embodiments, the subject is a mammal, and the mammal is a mouse or a human. In certain embodiments, the administration to the subject is performed via intravitreous, intrathecal, intramuscular, intradermal, intracranial, intraspinal, or epidural injection. In certain embodiments, the hydrogel is administrated to a subject affected by a CNS disorder or injury. In certain embodiments, the subject is affected by a CNS disorder or injury, including, but not limited to, spinal cord injury, traumatic brain injury, stroke, glaucoma, optic nerve injury, retinal tissue injury or disorder, muscle dystrophy, muscle hypertrophy, metabolic myopathies, or muscle paralysis. In certain embodiments, spidroin-SpyTag in hydrogel form covalently conjugated with one or more protein therapeutics, is injected in a targeted site affected by a CNS disorder or injury to promote neuroprotection and axon regeneration. In one example, the site of injury comprises an optic nerve and/or a retinal tissue.

In certain embodiments, the stiffness level of the spidroin-SpyT ag hydrogel can be tailored by adjusting protein concentration of the protein therapeutic covalently conjugated to the spidroin-SpyTag. In preferred embodiments, the stiffness level of the spidroin-SpyT ag hydrogel covalently conjugated to protein therapeutics is comparable to the stiffness of neural tissue.

In certain embodiments, the covalent immobilization or conjugation of protein therapeutics to the spidroin-SpyTag hydrogel allows the slow sustained release of the protein therapeutics in vitro or in vivo. In certain embodiments, the method of the subject invention can be used for culturing neurons in a cellular system for studying axon regeneration. In certain embodiments, the spidroin-SpyTag hydrogel conjugated to protein therapeutics can be used as a substrate for the attachment of primary dorsal root ganglion (DRG) neurons. In some examples, the simultaneous release of CNTF from the hydrogel to cultured dorsal root (DRG) neurons promotes neurite growth by triggering the JAK-STAT3 signaling pathway.

In certain embodiments, the spidroin-SpyT ag hydrogel is stable in a target site for up to 14 days, preferably up to 30 days or more.

In certain embodiments, the hydrogel comprising the spidroin-SpyTag covalently conjugated with one or more protein therapeutics is administered to the subject via intravitreous, intrathecal, intramuscular, intradermal, intracranial, intraspinal, or epidural injection.

In some embodiments of the invention, the method comprises administration of multiple doses of the compositions of the subject invention. The method may comprise administration of therapeutically effective doses of a composition comprising the compound or composition thereof of the subject invention as described herein once a week, once a month, once a quarter, twice a year, once a year, or a lower frequency. Moreover, treatment of a subject with a therapeutically effective amount of the compositions of the invention can include a single treatment or can include a series of treatments. It will also be appreciated that the effective dosage of a compound or composition thereof used for treatment may increase or decrease over the course of a particular treatment. Changes in dosage may result and become apparent from the results of diagnostic assays determine the restoration of neural function (or absence of), which are known in the art. Specifically, the identification of nerve regeneration includes, for example, motor skills evaluation.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims. All references cited herein are hereby incorporated by reference.

The concentration/amount of active agent(s) in a formulation can vary widely, and will be selected primarily based on activity of the active ingredient(s) in accordance with the particular mode of administration selected and the patient's needs. Concentrations, however, will typically be selected to provide dosages ranging from about 1-30 μM. It will be appreciated that such dosages may be varied to optimize a therapeutic and/or prophylactic regimen in a particular subject or group of subjects.

It should be apparent to one skilled in the art that the exact dosage and frequency of administration will depend on the particular condition being treated, the severity of the condition being treated, the age, weight, general physical condition of the particular patient, and other medication the individual may be taking as is well known to administering physicians who are skilled in this art.

### Materials and Methods

### Construction and Production of Spidroin-SpyTag

The gene and amino acid sequences of NT D 2RepCTD-SpyTag (S-A) are detailed in Table 1 and 2. This construct was cloned into a pET22b (+) vector and subsequently introduced into Escherichia coli BL21 (DE3) cells (Invitrogen) through transformation. The cells were cultured in Luria broth (LB) supplemented with 100 mg/L ampicillin at 37° C. and 220 rpm until the optical density at 600 nm (OD 600) reached a range of 0.6 to 1.0. To induce protein expression, 3.5 mM isopropyl β-D-1-thiogalactopyranoside (IPTG, Sangon Biotech) was added to the culture. Following induction, the cells were incubated at 16° C. for 20 hours, after which they were harvested and resuspended in 20 mM Tris-HCl (pH 8). To prevent protein degradation, phenylmethylsulfonyl fluoride (1 mM) was added to the resuspended cells, and then the cells were lysed using a French Press cell crusher. The lysate was centrifuged at 18,000×g at 4° C. for 45 minutes and the resulting supernatant was filtered through a 0.45-μm filter. The filtered supernatant was loaded onto Ni-NTA columns (Cytiva) and washed with a buffer containing 20 mM Tris-HCl and 20 mM imidazole (pH 8). Finally, the target proteins were eluted using an AKTA Explorer liquid chromatographic system (GE Healthcare) with an elution buffer containing 20 mM Tris-HCl and 500 mM imidazole (pH 8).

The protein solution obtained was subjected to dialysis against 20 mM Tris-HCl (pH 8) at 4° C., employing a total volume of 5 liters divided into six cycles. Subsequently, the solution was filtered using a 0.22 μm filter and concentrated to a final concentration of 40 mg/mL using Amicon Ultra-15 centrifugal filters (Millipore). The protein concentration was determined by measuring the UV absorbance at 280 nm, while SDS-polyacrylamide gel electrophoresis and Coomassie Brilliant Blue staining were employed to assess the purity. The expression yield of S-A was approximately 40 mg per liter of E. coli culture. For future use, the S-A solution was stored either at 4° C. or −80° C.

### Construction and Production of SpyCatcher-Fusion Proteins

We created three expression systems, pQE801::B-CNTF-B, PQE801::B-IGF1-B and pQE801::B-OPN-B, to produce SpyCatcher-fusion proteins including SpyCatcher-ELP-CNTF-ELP-SpyCatcher, SpyCatcher-ELP-Osteopontin-ELP-SpyCatcher, and SpyCatcher-ELP-IGF1-ELP-SpyCatcher. They were constructed by inserting the respective gene encoding CNTF, Osteopontin, or IGF1, into the previously described plamid, pQE801::SpyCatcher-ELP-RGD-ELP-SpyCatcher39, using SacI and SpeI restriction enzymes. The corresponding proteins were expressed using E. coli BL21 (DE3) and subsequently purified via Ni-NTA affinity chromatography. The purified proteins were dialyzed against Milli-Q® water (5 liters×6) at 4° C. and then sterilized using a 0.22 μm filter. Finally, the proteins were lyophilized using a Labconco lyophilizer and stored at −80° C. for future use.

### Preparation of Spidroin-SpyTag Hydrogel

We utilized a Branson SFX 250 Sonifier (250 W, 20 kHz) equipped with a 3-mm-diameter conical microtip. In a typical process, 0.5 mL of S-A solution in a 1.5-mL Eppendorf tube was subjected to pulsed sonication (20 cycles of 1 second on, 5 seconds off) at an amplitude of 20% at room temperature. To ensure sterilization and prevent overheating of the protein solution, 75% Ethanol was utilized. Minimize the introduction of air bubbles during sonication. The resulting sonicated solution was referred to as the pre-gel solution. To initiate gelation, the sonicated protein solution was placed at 37° C.

### Rheological Tests

An ARES-RFS rheometer (TA Instruments) was utilized to perform rheological measurements in time-, frequency-, and strain-sweep modes. The rheometer setup involved a bottom steel plate with a diameter of 25 mm, with the sample being placed at the center of the plate. On top, there was an 8 mm diameter steel plate, and the distance between the top and bottom plates was fixed at 0.5 mm. All experiments were conducted at 37° C. To mitigate water evaporation, the sample was sealed with silicone oil. Gelation kinetics were monitored through time-sweep tests, with the strain and frequency fixed at 5% and 1 rad/s, respectively. Frequency-sweep tests were performed over a frequency range of 0.01-100 rad/s, with the strain fixed at 5%. Strain-sweep tests were performed over a strain range of 1-250% at a constant frequency of 1 rad/s.

### Erosion Tests

To evaluate the erosion of the S-A hydrogels, 30 μl of 4 wt % hydrogel samples were submerged in 0.5 ml of Tris buffer, PBS, or milliQ® water. At designated time points, 2 μl aliquots of the supernatant were withdrawn, and the absorbance at 280 nm was measured using a NanoDrop 2000c spectrophotometer (Thermo Scientific) to determine protein concentration. The experiment was performed in triplicate. The erosion percentage was calculated as follows:

Percentage of erosion=(the amount of protein in supernatant/the total amount of protein in gel)×100%

### Circular Dichroism (CD) Spectroscopy

CD measurements were performed using a Jasco-8815 CD spectrophotometer (Jasco Co.) at room temperature. To assess the alterations in secondary structures, the ellipticity values of the S-A solution, before and after sonication, were recorded. A 0.2 mL aliquot of the S-A solution (5 μM) in Tris buffer (pH 8) was dispensed into a quartz cuvette. Subsequently, samples were scanned across a wavelength range of 260 to 190 nm using the following settings: Continuous scan mode, scanning speed of 20 nm/min, and an accumulation of 1.

### Transmission Electron Microscopy (TEM) Analysis

Formvar-coated copper grids were negatively charged. Three microliters of the sample were gently deposited onto a grid, and any excess sample was carefully removed using blotting paper. Subsequently, the samples were washed with 2% (w/v) uranyl formate to enhance contrast. Following the wash, the grid was stained with uranyl formate for 45 seconds. Any excess stain was then removed using blotting paper, and the grid was allowed to air dry. Imaging was carried out using a Talos120c microscope operated at 120 k eV. Images were captured at magnification of ×22,000 or ×73,000.

### Covalent Conjugation Via Spy Chemistry

To examine the covalent conjugation via Spy chemistry, spidroin-SpyTag (S-A, 3 μg/μl) was mixed with SpyCatcher-POI-SpyCatcher (B-POI-B, 1 μg/μl) at a molar ratio ranging from 4:1 to 6:1. The mixtures were then incubated at 4° C. overnight, followed by SDS-PAGE analyses. The use of excess spidroin-SpyTag was intended to ensure the completion of the reactions and the depletion of the other reactant, B-POI-B, to simplify subsequent SDS-PAGE analyses.

### Protein Release Assays

The sonicated S-A solution (3 wt %) was mixed with either B-GFP-B or A-GFP-A (1 μg/μl) and then incubated at 4° C. overnight. Afterward, 60 μl aliquots of the reaction products were transferred into 1.5 mL Eppendorf tubes and subjected to incubation at 37° C. for 1 hour to facilitate hydrogel formation. To evaluate the release of GFP, 100 μl of 20 mM Tris-HCl buffer (pH 8.0) was added to each tube. These tubes were then placed in a 37° C. humidified incubator. After 1 and 3 days, 100 μl aliquots of the supernatant were transferred to a black 96-well plate (Nunc). The fluorescence intensity of the supernatant was measured using a Varioskan LUX multimode microplate reader (ThermoScientific) with excitation at 470 nm and emission at 510 nm. The ratio of released GFP was calculated as follows:

$$Ratio of released G F P ( \% ) = ( Fluorescence intensity of 100 µl of supernatant / Fluorescence intensity of 100 µl of respective control ) \times 100 \%$$

In this equation, “the respective control” refers to the gelation precursor (60 μl), S-A+B-GFP-B (or A-GFP-A), that was diluted with 100 μl of 20 mM Tris-HCl buffer (pH 8.0) to mimic the 100% release of GFP from the gel to the supernatant.

### Covalent Functionalization of S-A Hydrogels

The sonicated S-A solution was mixed with B-POI-B and the reaction mixture was placed at 4° C. overnight. Gelation was initiated by moving the solutions to 37° C. or injecting them into mice. The amounts of CNTF used in vitro and in vivo were primarily determined by pre-screening different concentrations under the respective conditions. In the in vitro assays, the concentrations of B-CNTF-B varied, with 4 μg/μl (i.e., 60 μM) used for the N2A cell experiments and 8 μg/μl (i.e., 120 μM) for the DRG neuron studies. This variability is likely attributable to the differing sensitivities of N2A and DRG cells to the neurotrophin. In all in vivo experiment, a consistent concentration of 1 μg/μl (i.e., 15 μM) B-CNTF-B was employed.

### Culturing of N2A Cells

N2A cells (ATCC, Cat #CCL-131, RRID: CVCL_0470) were cultured in high-glucose DMEM (Gibco) supplemented with 10% (v/v) fetal bovine serum (FBS) (Gibco) and 1% (v/v) penicillin-streptomycin (Gibco). The cells were cultured in a CelCulture CO2 incubator (Esco Micro Pte. Ltd.) under conditions of 37° C. and a 5% CO2 atmosphere, with regular passaging every three days. When the cells reached 70 to 80% confluence, they were detached using 2 to 3 ml of TrypLE (Gibco). Subsequently, 10 ml of complete medium was added to neutralize the TrypLE and stop the digestion process. Cells were seeded onto confocal dishes coated with blank S-A gel (4 wt %), B-CNTF-B gel (S-A+60 UM B-CNTF-B), or B-LM-B gel (S-A+80 UM B-LM-B). To coat a confocal dish (SPL) with functionalized S-A hydrogels for cell culturing purposes, 120 μl of sonicated S-A solutions with or without B-POI-B was gently dispensed onto the middle area of the dish, followed by curing at 37° C. for 1 hour.

The seeded cells were cultured in an incubator for either 1 day or 3 days. Cell viability was evaluated using a LIVE/DEAD® viability kit (Invitrogen) following the guidelines provided by the manufacturer. Before staining, DM EM was removed and no fixation step was performed, to ensure that only cells attached to the gels were stained. To visualize the cells, a confocal microscope (Nikon) was employed. In each experiment, we randomly captured and analyzed one image per condition, conducting a total of five independent experiments for each group (n=5).

### Culturing of Primary DRG Neurons

Adult DRG neurons were prepared following a previously established protocol [33]. Briefly, adult mice were euthanized, and L4-L6 DRGs were excised from both sides. These excised DRGs were then enzymatically dissociated using 0.5 mg/ml collagenase P (Roche) for 1.5 hours at 37° C. Subsequently, the collagenase-containing medium was substituted with Neurobasal A, and the DRGs were gently dissociated by pipetting 20 times using 1 ml pipette tips. These cells were plated on hydrogel-coated or PDL/Laminin-coated confocal dishes.

The hydrogel-coated dishes were prepared following the previously described method. S-A gel (4 wt %), B-LM-B gel (S-A+80 UM B-LM-B), B-CNTF-B gel (S-A+120 UM B-CNTF-B) were coated on the hole of confocal dishes. As a positive control, the confocal dishes were initially treated with a poly-D-lysine solution (PDL, Sigma) at a concentration of 100 μg/ml, and incubated overnight at 37° C. The following day, the PDL solution was removed by aspiration, and the confocal dishes were washed five times with sterile water. Subsequently, a laminin solution (Gibco) at a concentration of 10 μg/ml was added and incubated at 37° C. for 2 hours. Finally, the laminin solution was aspirated, and the confocal dish was rinsed with 1×PBS. The neurons on different substrates were maintained at 37° C. and 5% CO2. The culture medium consisted of Neurobasal-A (Gibco) supplemented with 2% B 27 (Gibco) and 1% L-Glutamine (Gibco).

### Immunostaining and Quantification of Primary DRG Neurons

After 16 hours of incubation, the cells were fixed with 4% paraformaldehyde solution (Sigma-Aldrich) at room temperature for 10 minutes. Blocking and permeabilization were achieved using a solution containing 0.1% TritonX-100 (Sigma-Aldrich) and 4% normal goat serum (Invitrogen) at room temperature for half an hour. Subsequently, the cells were incubated with the primary antibody (Rb-TUJ1, Biolegend) diluted in 4% NGS at 4° C. overnight. The cells were then washed three times with 1×PBS and incubated with the secondary antibody Goat anti-Rabbit 488 (Invitrogen) at room temperature for 1.5 hours. After three washes with 1×PBS, the cells were maintained in PBS at 4° C. until further analysis. Images of the stained cells were captured using a laser scanning confocal microscope (Leica SP8).

To determine the cell density, three independent experiments were performed using primary DRG neurons from three different donors (n=3). In each experiment, at least 256 DRG neurons were counted per condition to ensure statistical rigor. Neurite outgrowth was measured and quantified using the NeuronJ plugin within ImageJ. Three independent experiments were performed. In each experiment, the average neurite lengths per DRG neuron was determined by analyzing at least 15 DRG neurons per condition, corresponding to 7 immunofluorescence images per dish.

### Animals and Surgeries

The study employed 6-8-week-old C57B L/6J mice (Charles River), adhering strictly to the guidelines established by the Laboratory Animal Facility at the Hong Kong University of Science and Technology and under the animal license number of Dr. Chao Yang: DH/HT&A/8/2/2 Pt.9. Prior to surgery, the mice were separated randomly into different groups and anesthetized using a combination of ketamine (80 mg/kg) and xylazine (10 mg/kg). With the eye exposed using an artery clamp, a meticulous incision was made in the conjunctiva using scissors to expose the optic nerve. Subsequently, the optic nerve was gently crushed with forceps (Dumont #2 and #5, Fine Science Tools). Following this, a Hamilton syringe was used to withdraw 2 μl of the vitreous body, and 2 μl of vehicle, blank S-A gel, B-CNTF-B solution (15 μM), B-CNTF-B gel (S-A+15 UM B-CNTF-B), or CNTF gel (S-A+15 μM CNTF, purchased from Alomone Labs, was injected into the eye. Post-operatively, eye ointment was applied to prevent infection, and ketoprofen (0.05 ml/kg) was injected for analgesia. Two days prior to sacrificing the animal, 2 μl of CTB-FITC (1 μg/μl, Sigma-Aldrich) was injected intravitreally to label the axons.

### Immunostaining of Retinas and Optic Nerves

The mice were anesthetized with a lethal dose of ketamine/xylazine and subsequently perfused with PBS and 4% paraformaldehyde (PFA). The eyes and optic nerves were excised and postfixed overnight in 4% PFA prior to dissection for staining procedures. Whole-mount Tuj1 (Biolegend) staining of the retinas was performed to assess the survival of RGCs. Cryosectioning of the optic nerves and retinas was conducted, followed by immunostaining to detect regenerated axons (FITC antibody. Invitrogen), phosphorylated STAT3 (p-STAT3)-positive RGCs (Cell Signaling), Iba1-positive microglia (Wako Chemicals USA), CD4-positive helper T cells (BioLegend), CD68-positive activated macrophages and microglia (Bio-Rad), and CD45-positive hematopoietic cells (BioLegend). Specifically, the retina and optic nerve sections were first cryoprotected in 30% sucrose overnight and then embedded in OCT compound (SAKURA). The sections were cut to a thickness of 25 μm for the retina and 8 μm for the optic nerve. Subsequently, the dissected retina tissue or section samples were blocked with 0.1% Triton X-100 in 4% normal goat serum for 30 minutes, followed by incubation with primary antibodies overnight. After incubation, the samples were washed with PBS and incubated with secondary antibodies for 1 hour. Finally, the samples were washed with PBS and mounted for imaging using a confocal microscope (Zeiss).

### Quantification

At least 10 retina section images (20× objective) were taken from each mouse after staining with cell markers for immune cell quantifications. Cells positive for Iba1, CD68, CD45, or CD4 were counted in all retina layers and then normalized by dividing the cell count by the measured area to determine the cell density per mm². At least 163 Iba1+ cells, 93 CD68+ cells, 106 CD45+ cells, and 10 CD4+ cells were counted per mouse, with 3-4 mice per condition for quantification, as noted in the figure legends.

For p-STAT3-positive RGCs quantification, RGCs displaying nucleus accumulation of bright p-STAT3 signal were identified as positive cells. The positivity rates were calculated by dividing the number of p-STAT3-positive RGCs by the total number of Tuj1-positive RGCs. A minimum of 10 retinal section images (63× objective) were acquired, with an average of 73 RGCs analyzed per mouse to calculate the percentage of p-STAT3-positive RGCs.

Whole-mount-stained retinas were imaged using a confocal microscope to quantify the surviving RGCs. Twelve images were captured from different regions of each retina. Tuj1-positive RGCs per mm² were then counted randomly to avoid biases, with at least 212 RGCs counted from each mouse. The RGC survival rate (%) after injury in different groups was calculated using the following formula:

$$R G C survival rate ( \% ) = ( number of surviving R G Cs in each group / average number of R G Cs in the uninjured control group ) \times 100 \%$$

The average number of RGCs in six uninjured mice was (2952±239) per mm².

To quantify the number of regenerated axons, the optic nerve was longitudinally cryosectioned at a thickness of 8 μm. The CTB-FITC signal was amplified through immunostaining with an FITC antibody and a secondary antibody (goat anti-rabbit, Alexa Fluor™ 555; Invitrogen). Regenerated axons were quantified by capturing five images from each optic nerve. The axon count was determined using the formula:

$$axon number = π {r}^{2} \times ( n / d ) / 8 µm ,$$

where r represents the radius of the optic nerve, n/d is the average axon count per average nerve width at the designated counting site, and the section thickness is 8 μm.

### Animal Behavior

Five C57B L/6 mice received vehicle intravitreal injections in both eyes, while another five received S-A gel intravitreal injections in both eyes. Animal behavior was monitored for 10 minutes at 1, 5, and 14 days post-injection using an overhead camera. After recording, the videos were analyzed by an observer who was blind to the treatment to determine if the mice displayed eye-rubbing behavior or any signs of distress.

### Statistical Analysis

Unless otherwise specified, data are expressed as mean±standard deviation (SD). Sample sizes (n) for each experiment are detailed in the corresponding figure legends and methods sections. Unpaired two-tailed t-tests were employed for comparisons between two groups. To compare results across multiple experimental groups, one-way or two-way ANOVA followed by post-hoc multiple analyses was conducted. Statistical significance was defined as *P≤0.05 and **P≤0.01 across all experiments. All data analyses were performed using GraphPad Prism 8.0 software.

Naturally occurring spidroins are noted for their robust phase transition from liquid condensates into solid fibers under physical stimuli. When it comes to injectable materials for in vivo therapeutic delivery, the most obvious stimulus from a biological system is the abrupt change of temperature, from the ambient (around or below ˜23° C.) to body temperature (˜37° C.). Spidroin-SpyTag protein (4%) after brief ultrasound treatment undergoes rapid sol-gel transition as the temperature elevates from room temperature to 37° C. This thermally triggered gelation confers injectability to the system.

By leveraging SpyTag/SpyCatcher chemistry, spidroin-SpyT ag can be covalently modified with various bioactive proteins, resulting in a versatile platform capable of sustained therapeutic release in vivo. To demonstrate the efficacy of this approach, we conducted a proof-of-principle experiment in which spidroin-SpyTag decorated with SpyCatcher-fusion ciliary neurotrophic factor (SpyCatcher-CNTF) was injected into the vitreous of mouse eyes. This intervention resulted in long-lasting neuroprotection and enhanced axon regrowth following optic nerve injury.

The findings from this study highlight the recombinant spider silk protein as a generalizable platform for injectable protein therapeutics. The unique properties of the protein, including its thermally induced phase transition behavior and genetically encoded click chemistry, hold immense potential for addressing a broad range of biomedical challenges beyond CNS axon regeneration.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1—Creation and Functionalization of a Novel, Injectable Spidroin Hydrogel

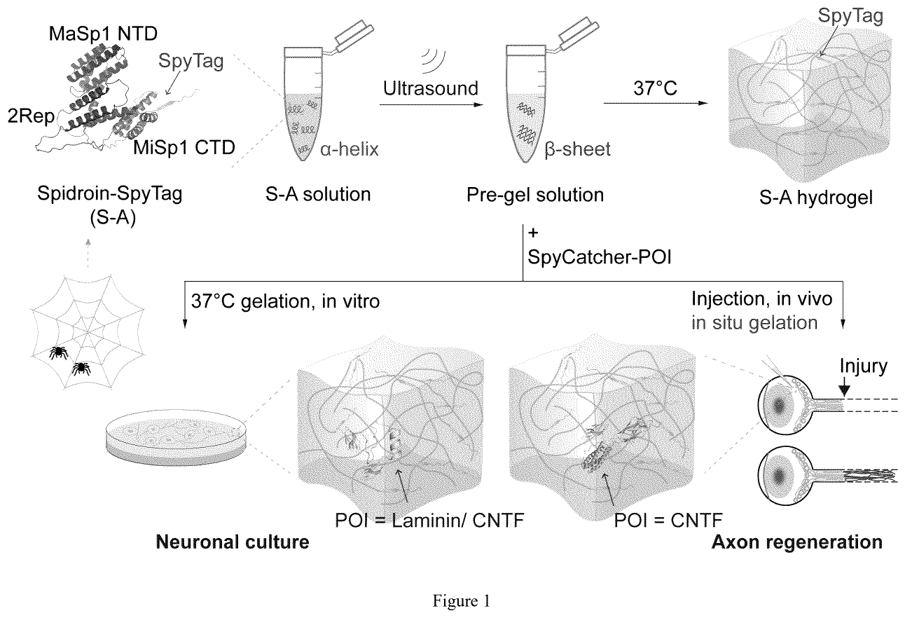

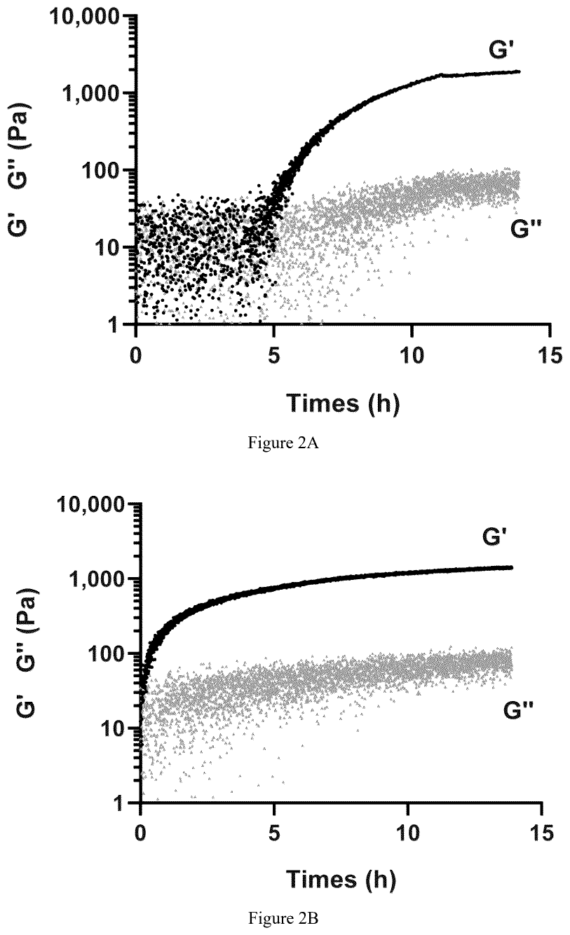

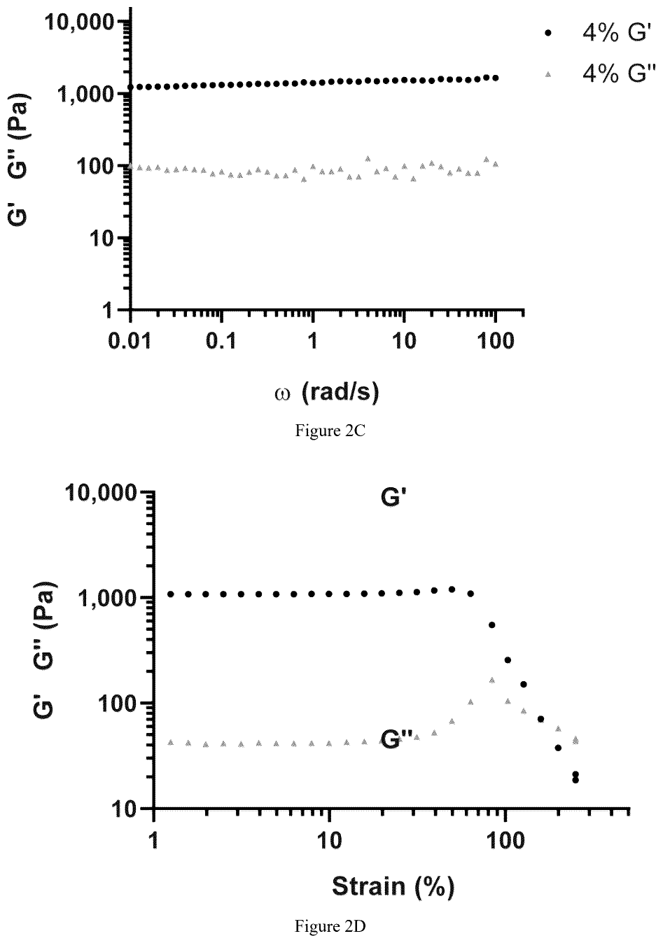

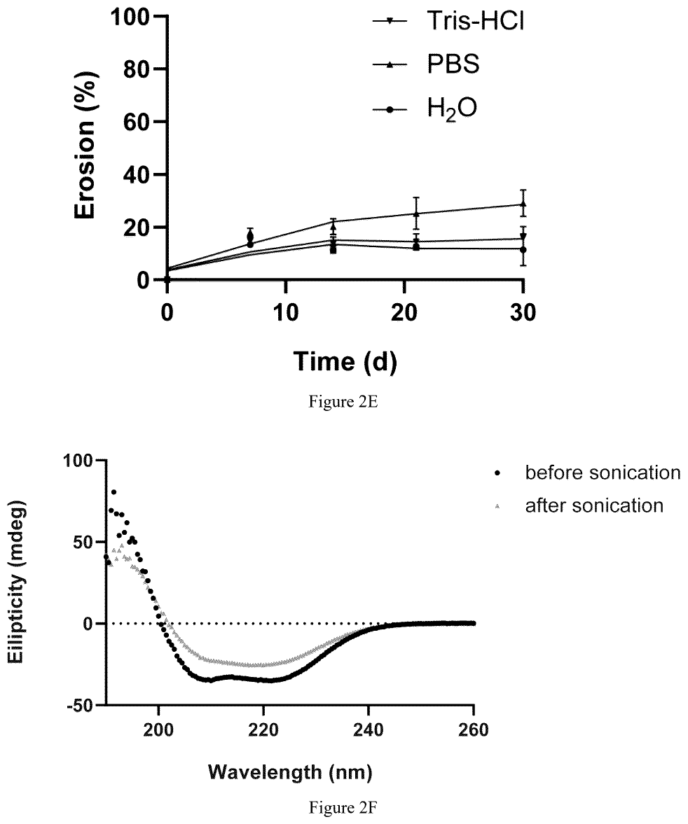

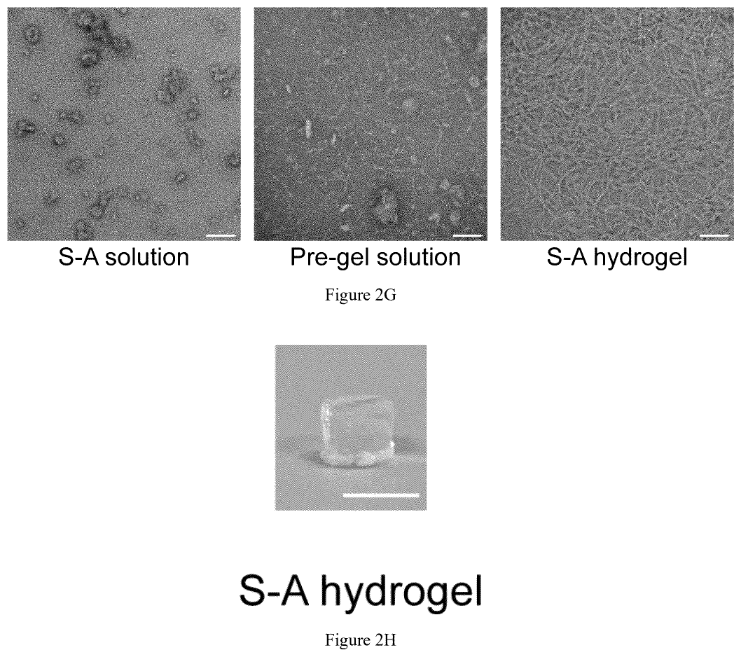

We designed and produced a recombinant spider silk protein called spidroin-SpyTag (S-A). The protein solution, when subjected to brief sonication, exhibits a rapid transition from sol to gel state at body temperature (37° C.), thereby enabling its injectability (FIGS. 1 and 2A-2H). Additionally, the stiffness of the resulting hydrogel can be tailored by adjusting the protein concentration. Hydrogels formed at 40 mg/mL (G′˜1.2 kPa) and 30 mg/mL (G′˜0.5 kPa) demonstrated stiffness levels comparable to neural tissue (G′˜0.15-1.5 kPa) [34, 35]. Moreover, the resulting S-A gels eroded less than 30% when exposed to various salt solutions for up to 30 days (FIGS. 2A-2H). We conducted preliminary investigations to understand the mechanism behind hydrogel formation. Circular dichroism (CD) spectroscopy and transmission electron microscopy (TEM) analyses revealed an increase in β-sheet content and the presence of enlarged and entangled protein fibrils (FIGS. 2A-2H), which are associated with the formation of the hydrogel.

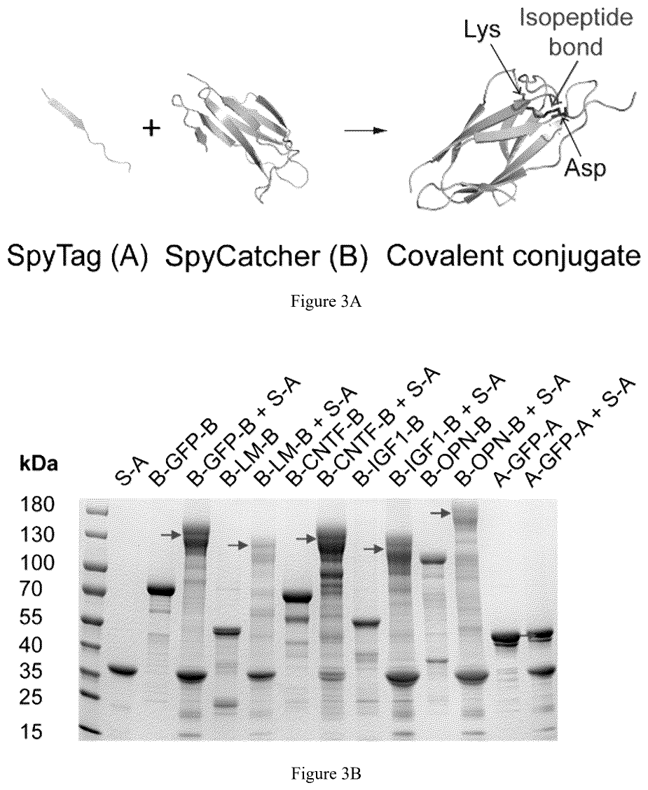

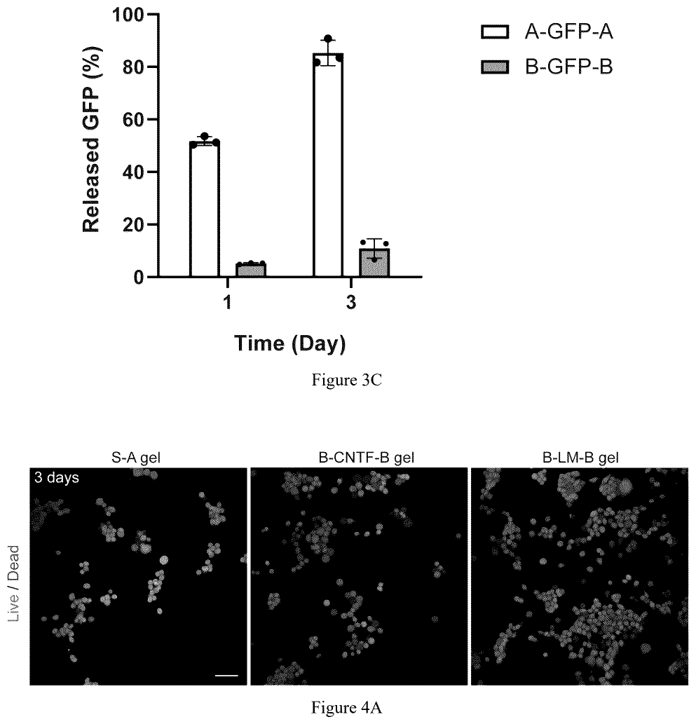

Another novel aspect of this invention is the incorporation of Spy chemistry into the system. The integration of Spy chemistry enables the functionalization of the hydrogel with a wide range of bioactive motifs, including CNTF, laminin, Insulin-like growth factor 1, and Osteopontin (FIGS. 3A-3C). The covalent immobilization of protein therapeutics onto the hydrogel network offers the potential for slow release kinetics and, consequently, more sustained effects in vivo compared to conventional physical encapsulation methods. This injectable material system, combined with its capacity for versatile protein modification, establishes a versatile platform for the delivery of protein therapeutics. Due to its broad applicability, this technology addresses various chronic diseases and injuries, extending beyond neuronal injury and neurodegenerative conditions.

### Example 2—Neuron Culturing by Spidroin-SpyT Ag Hydrogels

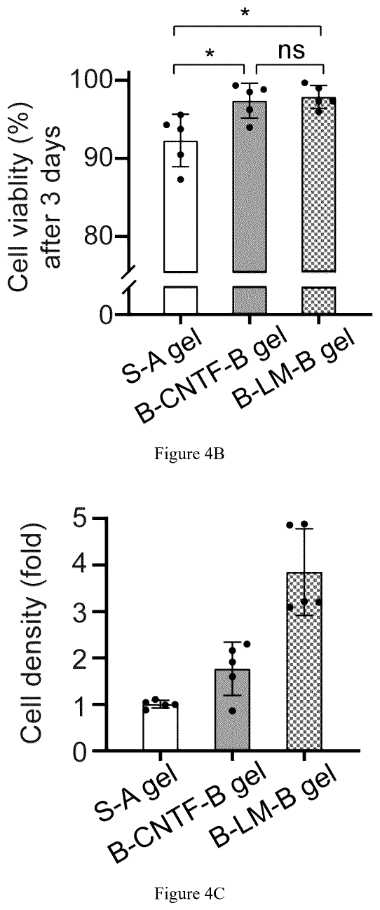

N2A cells were highly viable on the blank S-A hydrogel after 1 day. To improve the adhesion and viability of N2A cells, we created two novel functional hydrogels, namely B-CNTF-B gel and B-LM-B gel, by conjugating the gel precursor, S-A, with B-CNTF-B and B-LM-B, respectively. After 3 days, the cell viability on the blank S-A gel decreased slightly [(92±3) %], while remaining high on the B-CNTF-B and B-LM-B gels (˜97% and 98%, respectively). Furthermore, the cell densities observed on the B-CNTF-B and B-LM-B gels were four times and twice as high, respectively, compared to those on the S-A gel, indicating that the approach of immobilizing CNTF and laminin successfully facilitated cell growth and proliferation (FIGS. 4A-4C).

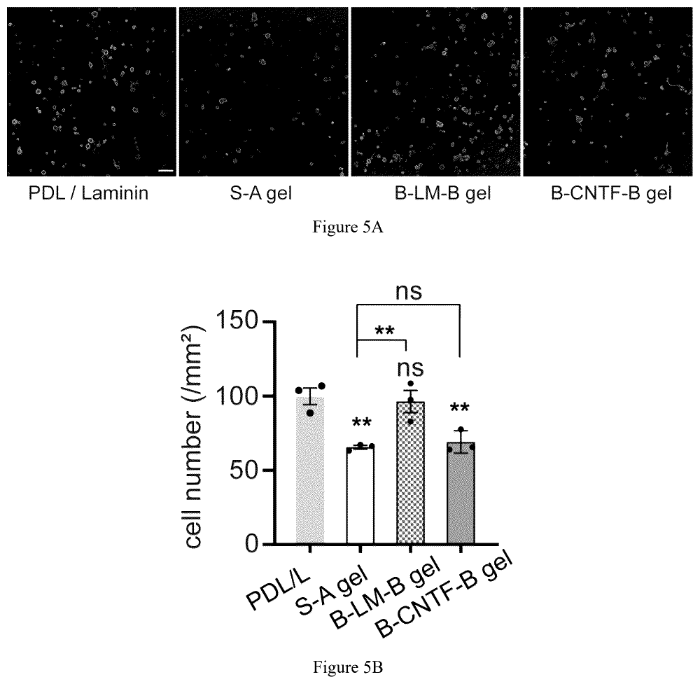

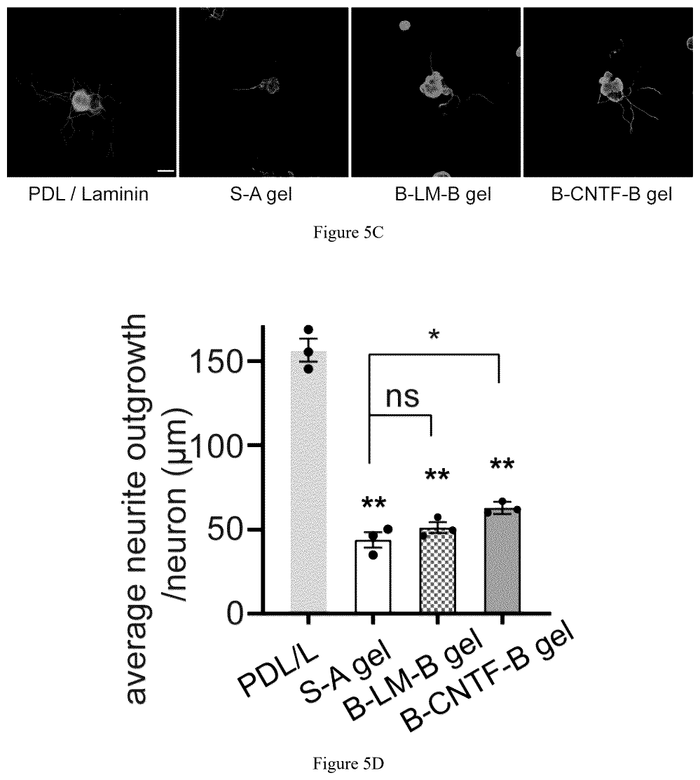

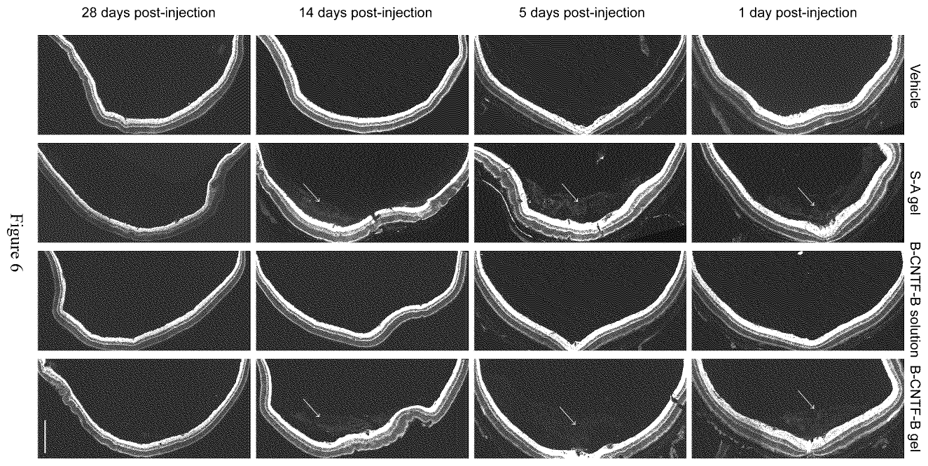

We also examined the suitability of these hydrogels for culturing dorsal root ganglion (DRG) neurons, a commonly used cellular system for studying axon regeneration [36]. The cell density on the coating comprising the laminin-laden gel turned out to be comparable to that on PDL/laminin (FIGS. 5A-5D), suggesting that S-A hydrogel decorated with laminin ligands via Spy chemistry is an ideal substrate in supporting the attachment of primary DRG neurons. In addition, while the cells exhibited only limited neurite growth on the blank S-A gel, augmented neurite growth (˜1.3 fold, FIGS. 5A-5D) was observed after covalently decorating the gel with CNTF, a neurotrophin that activates JAK-STAT3 and thus promotes neurite growth [37]. Together, these results demonstrated spidroin-SpyTag as a cytocompatible and versatile material system for encapsulating bioactive proteins, thereby enabling the modulation of neuronal behavior in vitro.

### Example 3—Degradability and Immunogenicity of Spidroin-SpyT Ag Hydrogels In Vivo

The S-A gel (3% wt) exhibited a modest degradation rate in vivo following intravitreal injection into the eye; at two weeks post-injection, histological analysis revealed the remaining materials within the vitreous body, distributed along the retinal surface, while after four weeks, the gels were completely degraded, while avoiding any noticeable retinal deformation (FIG. 6).

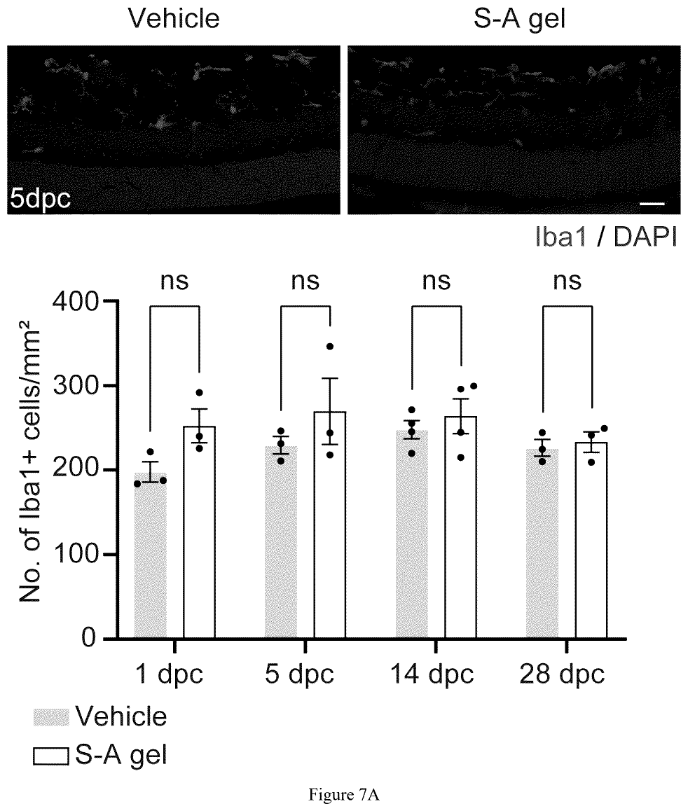

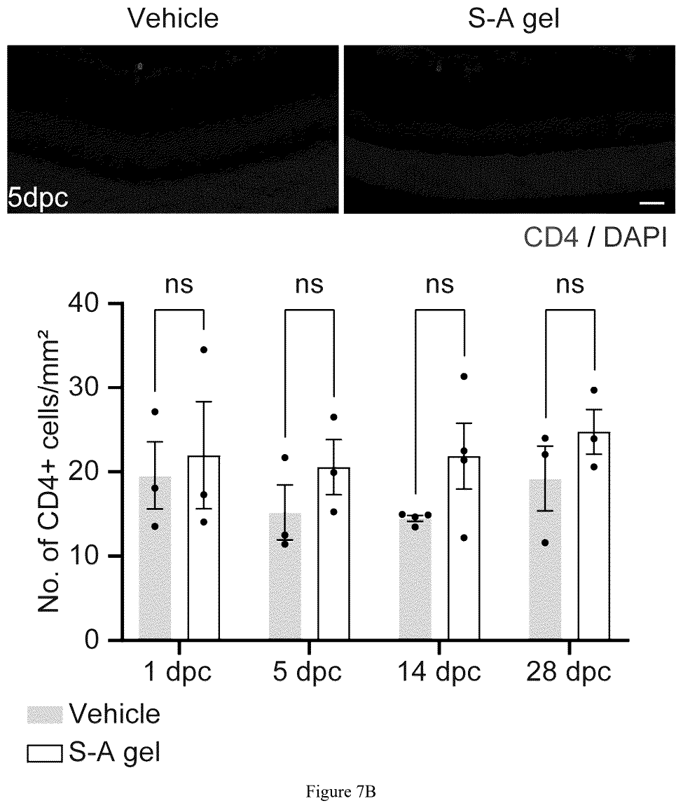

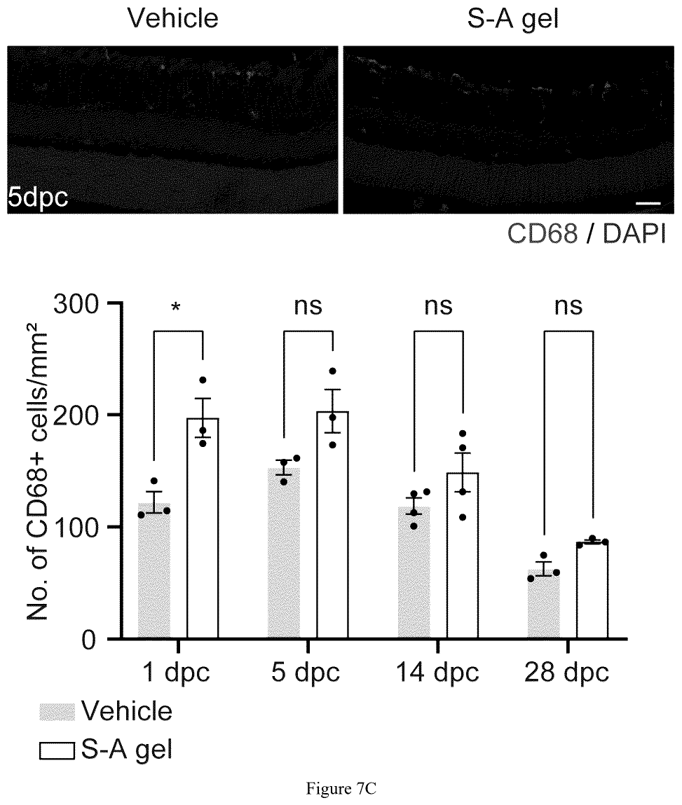

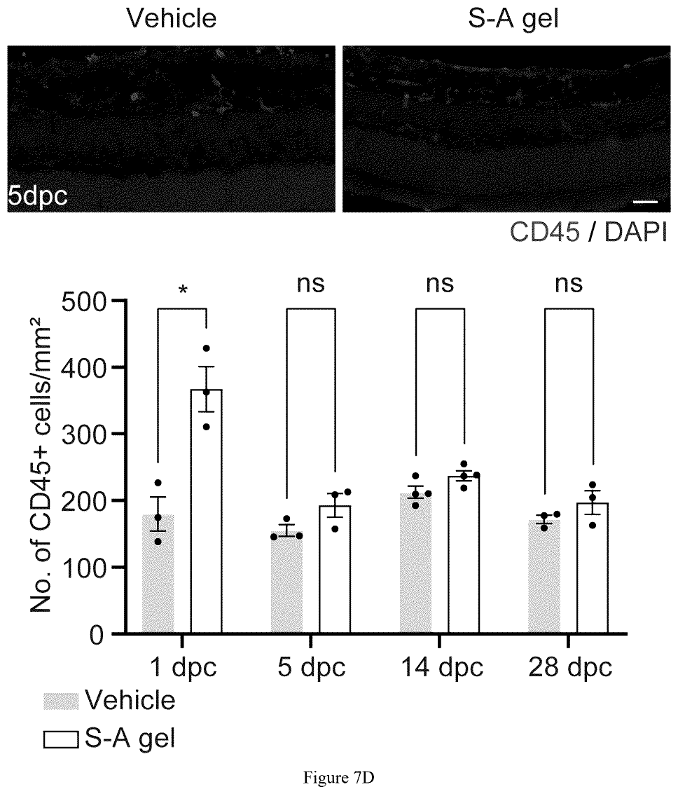

To assess their immunogenicity in the retina, we injected the vehicle or S-A gel directly into the vitreous body following optic nerve crush in C57BL/6 mice. Retinas were collected at 1, 5, 14, or 28 days post-injection, and stained for common immune cell markers to identify specific immune cell populations. The numbers of microglia (Iba1-positive) and helper T cells (CD4-positive) in the presence of the S-A gels were comparable to those treated with the vehicle at all time points (FIGS. 7A-7D). Furthermore, we observed a greater presence of activated macrophages and microglia (CD68-positive), and hematopoietic cells (CD45-positive) in samples treated with the S-A gel at 1 day post-injection. However, no significant differences were found in the two biomarkers of immune cells between the vehicle and S-A gel treatments at 5 days, 14 days, and 28 days post-injection (FIGS. 7A-7D).

We monitored animal behavior after S-A gel intravitreal injection into mice eyes to determine if these protein hydrogels induced any abnormal behaviors, such as eye-rubbing. Observations at 1, 5, and 14 days post-injection revealed that eye-rubbing was rare in mice injected with the hydrogels and comparable to those injected with the vehicle. Overall, the animals exhibited normal behavior after S-A gel injection and showed no signs of distress.

### Example 4—B-CNTF-B Spidroin Hydrogel for Prolonged STAT3 Activation

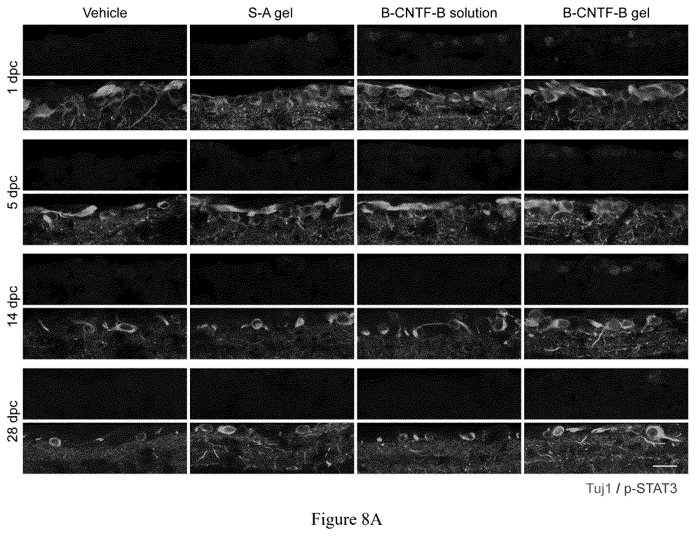

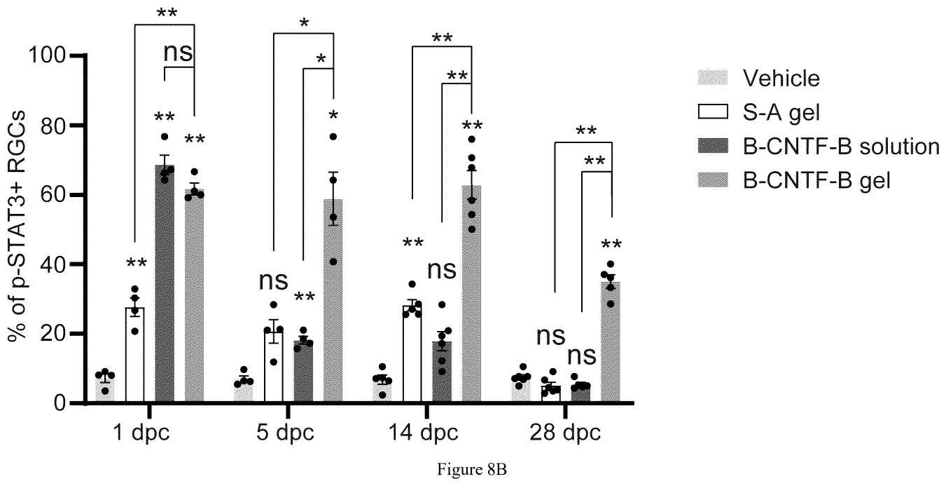

To assess the capability of the S-A hydrogel for prolonged CNTF signaling in vivo, we performed optic nerve crush in C57BL/6 mice and immediately administered either the vehicle (Tris buffer), blank S-A gel, B-CNTF-B solution (15 μM), or B-CNTF-B gel (S-A+15 UM B-CNTF-B) into the vitreous body. We then harvested the retinas at various time points post-injection (1, 5, 14, or 28 days) and immunostained them for phosphorylated STAT3 (p-STAT3). Quantitative analysis of p-STAT3-positive RGCs revealed that the vehicle induced minimal STAT3 phosphorylation (<10%) in RGCs one day after treatment, while both the B-CNTF-B solution and B-CNTF-B gel resulted in substantially increased levels of p-STAT3 (FIGS. 8A-8B). The discrepancy between the CNTF delivery methods became prominent with the passage of time. At 5 days post-injection, the mice treated with the B-CNTF-B solution exhibited a notable decrease in STAT3 phosphorylation (FIGS. 8A-8B). By contrast, the mice receiving the B-CNTF-B gel maintained persistently high levels of STAT3 phosphorylation, and approximately 60% of RGCs exhibited p-STAT3-positive over a two-week period (5 and 14 days) (FIGS. 8A-8B). Remarkably, even at four weeks post-injection, when the hydrogel had supposedly fully degraded, ˜35% of RGCs in the mice treated with the B-CNTF-B gel remained p-STAT3-positive, exceeding the other groups (FIGS. 8A-8B), which suggest that the spidroin-SpyTag gel can significantly extend the functional duration of CNTF in vivo. Unexpectedly, the administration of the blank S-A gel slightly augmented the level of phosphorylated STAT3 (p-STAT3) in RGCs after one day, perhaps because of an inherent ability of the spidroin materials to activate JAK-STAT3 (FIGS. 8A-8B). Collectively, our findings established the viability of using a thermally responsive protein material for sustained protein delivery and signaling in vivo. This approach circumvents the need for multiple injections when protein solutions are used and miti gates the risk of hyperactivating certain signaling pathways, a potential complication associated with viral delivery.

### Example 5—Enhanced Neuroprotection and Axon Regeneration by B-CNTF-B Hydrogel

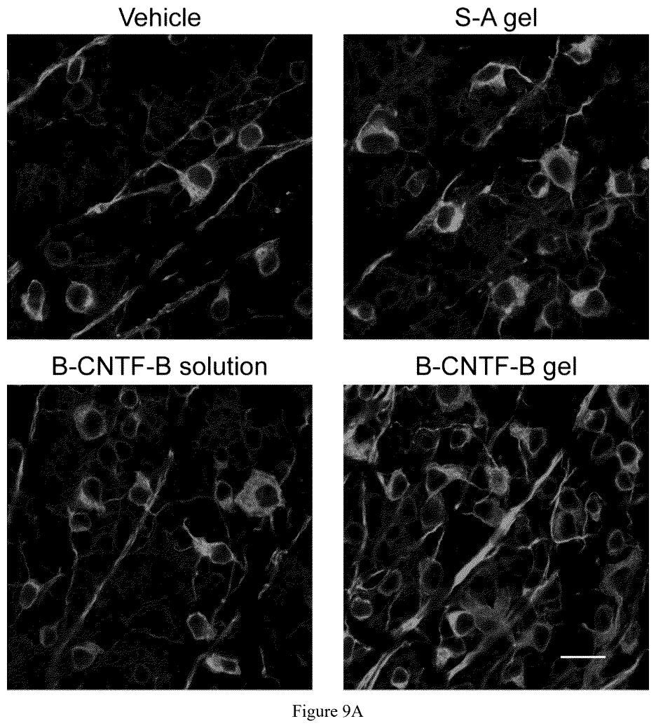

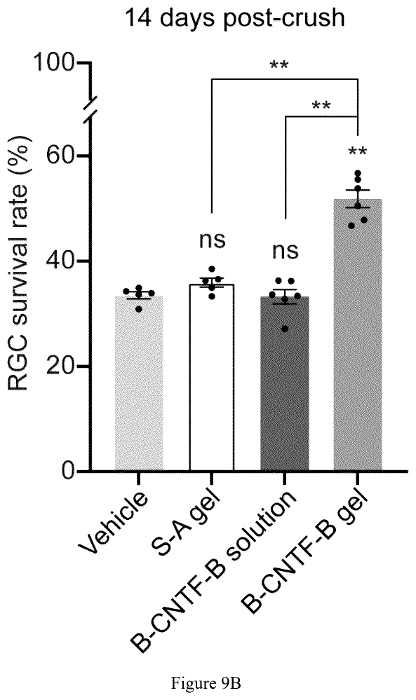

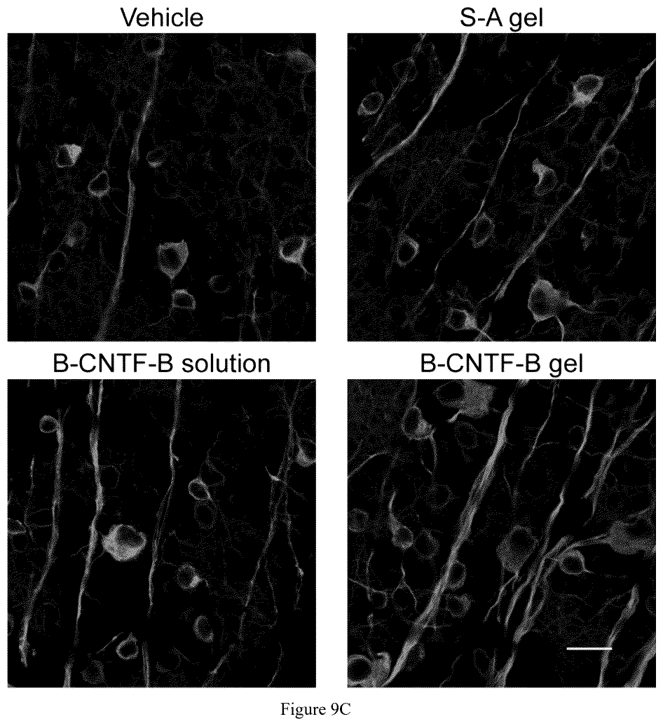

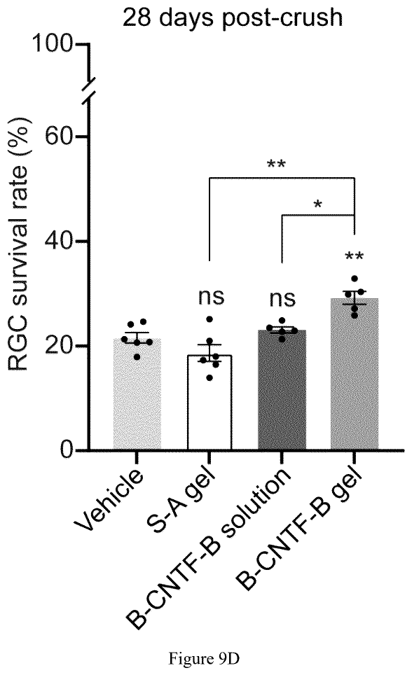

We employed the optic nerve crush model to assess the efficacy of spidroin-SpyTag hydrogel in promoting neuroprotection in vivo. Retinas and optic nerves were harvested at 2 and 4 weeks post-injury, with CTB-FITC injected intravitreally 2 days prior to sacrifice to label RGC axons. RGC survival was quantified using whole-mount Tuj1 staining. After two weeks, neither B-CNTF-B solution nor S-A gel treatments significantly protected RGCs. However, B-CNTF-B gel significantly enhanced RGC survival (FIGS. 9A-9D). After four weeks, when the gel had fully degraded, RGC survival decreased but remained significantly higher than under other conditions. These findings demonstrated the ability of B-CNTF-B gel to elicit sustained neuroprotection in vivo.

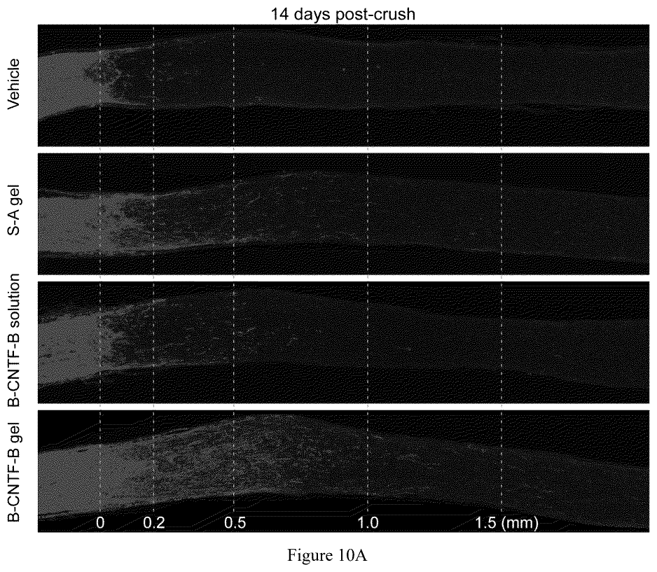

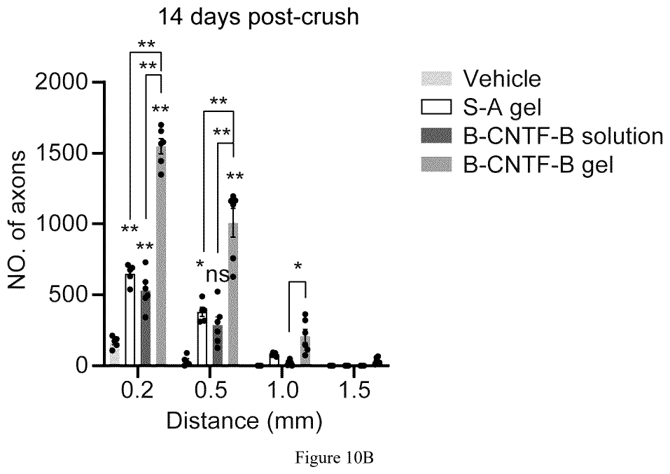

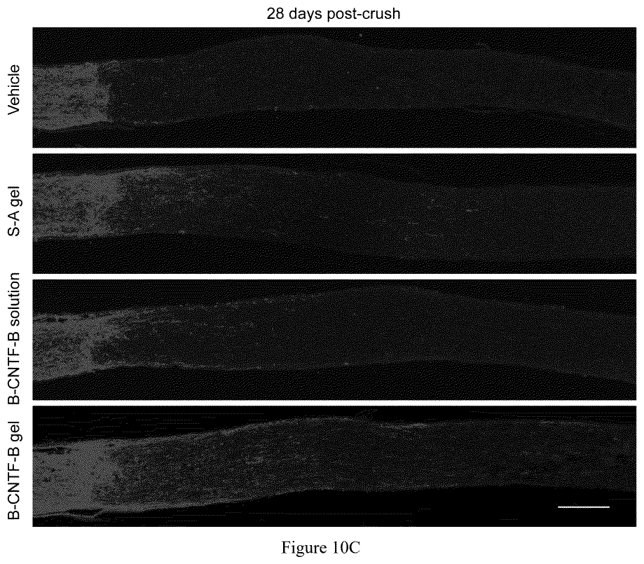

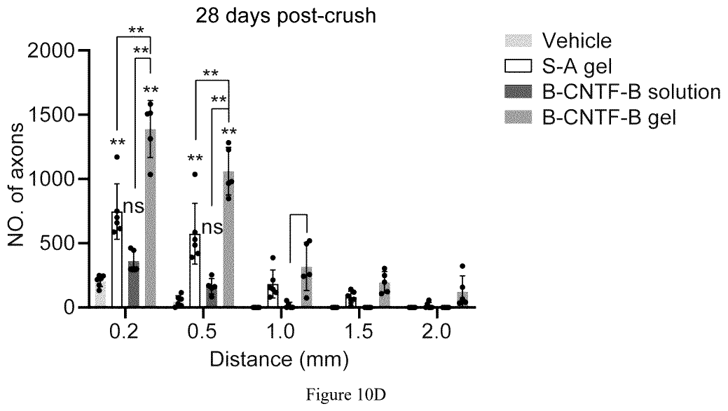

Injuries to adult CNS often result in permanent motor and sensory dysfunction. Among numerous factors involved, CNS axon regeneration is deemed crucial for functional recovery [36, 38]. We examined the impact of B-CNTF-B gel on axon regeneration within the injured optic nerve. In the control group treated with the Tris buffer, few axons regenerated across the lesion site at 2 weeks post-injury (FIGS. 10A-10D). The treatment with B-CNTF-B solution or with blank S-A gel modestly improved axon regeneration, with ˜530 and ˜650 axons extending longer than 0.2 mm, respectively. Without being bound to any theory, this may be due to short-term STAT3 activation by the B-CNTF-B solution or weak STAT3 activation by the blank gel (FIGS. 8A-8B). Notably, the B-CNTF-B gel significantly enhanced axon regeneration, with over 1500 axons regenerating across the lesion and longer than 0.2 mm, some even longer than 1.5 mm (FIGS. 10A-10D). At the 2-week time point, the B-CNTF-B gel was comparable to the adeno-associated virus-mediated genetic delivery of CNTF in promoting axon regeneration [33, 39]. At 4 weeks post-injury, the effect of the CNTF solution waned (FIGS. 10A-10D). In contrast, axon regeneration remained robust in the mice treated with the B-CNTF-B gel, with nearly 1400 axons regenerating more than 0.2 mm past the lesion site, and some regrowing up to 2 mm distal to the lesion (FIGS. 10A-10D). Interestingly, consistent axon regeneration, albeit modest, was observed in the optic nerves treated with the blank gels at both 2 and 4 weeks, pointing to the inherent capability of the recombinant spidroin protein in promoting axon regeneration. Taken together, these results demonstrated the feasibility of using spidroin-SpyTag as a simple, sustained protein delivery system to promote axon regeneration in optic nerves.

### Example 6—Covalent Immobilization is Essential for Sustaining CNTF Activity In Vivo

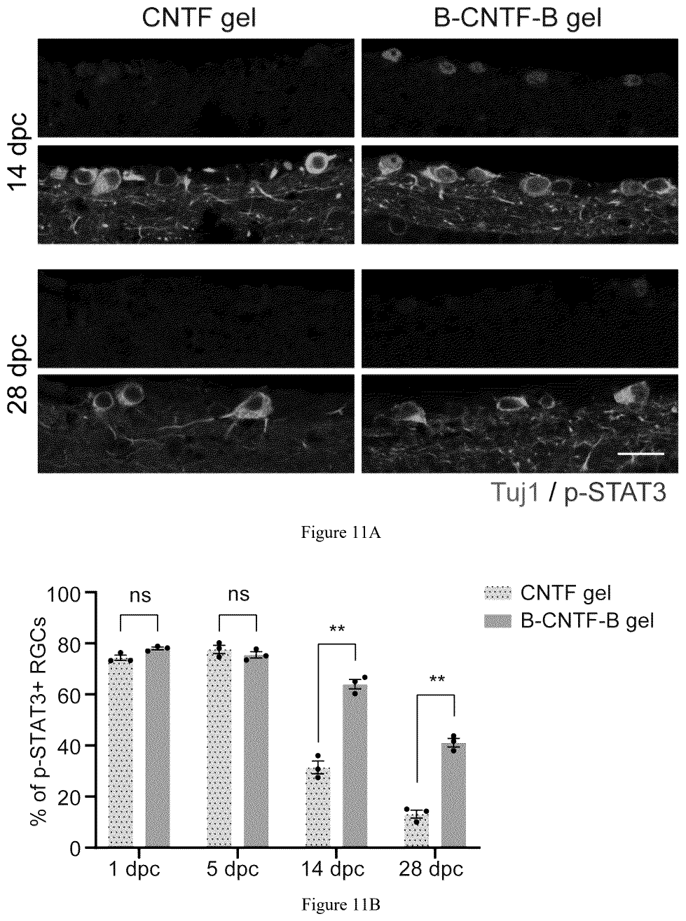

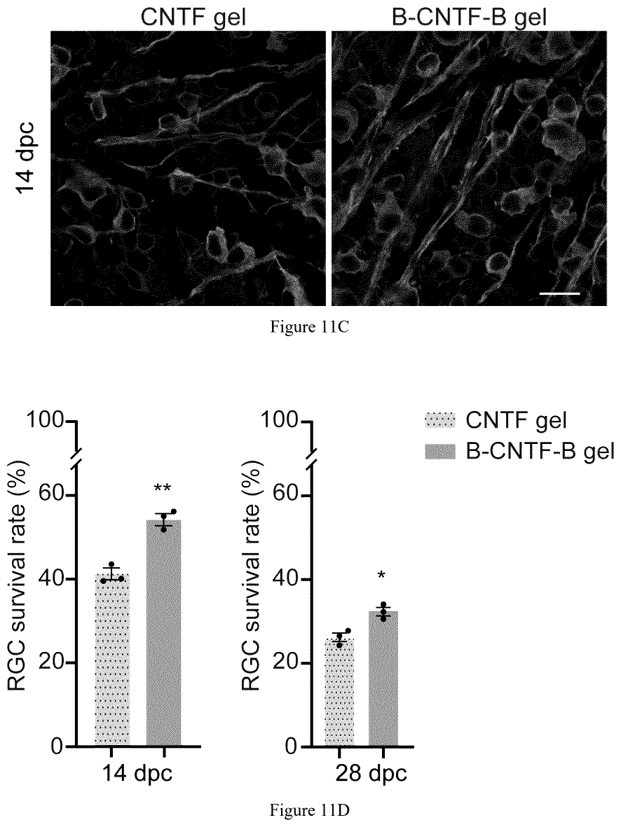

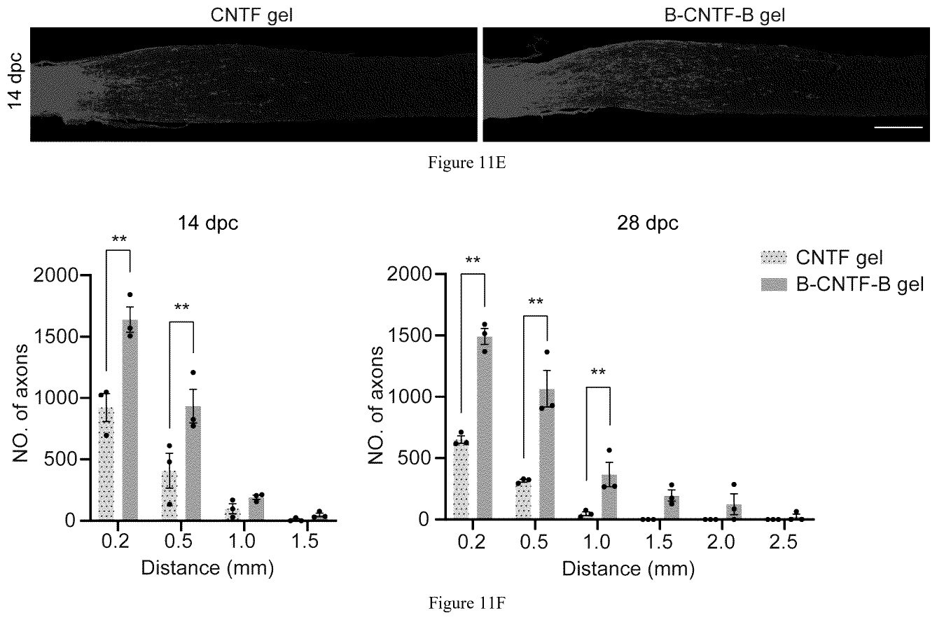

To further elucidate the role of covalent immobilization in CNTF delivery, we compared S-A hydrogels physically mixed with 15 μM CNTF (a commercial CNTF lacking the SpyCatcher (B) domain), i.e., CNTF gel, to the covalent B-CNTF-B gel, using the mouse model of optic nerve crush. While both systems elicited high levels of p-STAT3 at 1 and 5 days post-injection, with no apparent differences, they diverged significantly in STAT3 activation over a longer period (FIGS. 11A-11F). At 14 days post-injection, over 60% of RGCs were p-STAT3-positive in animals treated with covalently bound B-CNTF-B gel, compared to only 31% in those receiving physically mixed CNTF gels. This difference widened over time; at 28 days post-injection, 41% of RGCs in mice treated with the B-CNTF-B gel remained p-STAT3-positive, three times the levels observed in those treated with the CNTF gels (˜13%). Additionally, administration of the B-CNTF-B gel significantly enhanced RGC survival and axon regeneration at both 14 and 28 days post-injection, compared to the CNTF gels (FIGS. 11A-11F). These results point to the clear advantage of covalent immobilization in sustaining the activity of the neurotrophin.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### Exemplary Embodiments

Embodiment 1. An injectable protein delivery system, comprising recombinant spider silk protein spidroin-SpyTag, wherein the spidroin-SpyTag is in injectable hydrogel form, and wherein the spidroin-SpyTag is covalently conjugated to one or more bioactive agents.

Embodiment 2. The system of embodiment 1, wherein the one or more bioactive agents comprise one or more protein therapeutics.

Embodiment 3. The system of embodiment 2, wherein the one or more protein therapeutics comprise one or more of ciliary neurotrophic factor (CNTF), insulin-like growth factor (IGF1), or osteopontin (OPN).

Embodiment 4. A method for delivering a therapeutic agent to a central nervous system (CNS) targeted tissue, the method comprising administering an effective amount of the injectable protein delivery system of embodiment 1 to a targeted CNS tissue of a subject.

Embodiment 5. The method of embodiment 4, wherein the subject is a mammal.

Embodiment 6. The method of embodiment 5, wherein the mammal is a mouse or human.

Embodiment 7. The method of any of embodiments 4-6, wherein the spidroin-SpyTag transitions from sol to gel state after brief sonication and incubation at a temperature of about 37° C. for injectability.

Embodiment 8. The method of any of embodiments 4-7, wherein the spidroin-SpyTag is covalently conjugated with one or more protein therapeutics.

Embodiment 9. The method of embodiment 8, wherein the one or more protein therapeutics comprise one or more of ciliary neurotrophic factor (CNTF), insulin-like growth factor (IGF1), laminin, or osteopontin (OPN).

Embodiment 10. The method of any of embodiments 4-9, wherein an effective amount of the hydrogel comprising the spidroin-SpyTag covalently conjugated with one or more bioactive agents is administered to the subject via intravitreous, intrathecal, intramuscular, intradermal, intracranial, intraspinal, or epidural injection.

Embodiment 11. The method of embodiment 10, wherein the subject is affected by a CNS disorder or injury.

Embodiment 12. The method of embodiment 11, wherein the CNS disorder or injury comprises spinal cord injury, traumatic brain injury, stroke, glaucoma, muscle dystrophy, muscle hypertrophy, metabolic myopathies, or muscle paralysis.

Embodiment 13. The method of embodiment 11 or 12, wherein a site of the injury comprises an optic nerve and a retinal tissue.

Embodiment 14. The method of embodiment 12, wherein the hydrogel is injected in a CNS site of injury to promote neuroprotection and axon regeneration.

Embodiment 15. The method of embodiment 13, wherein the hydrogel is injected in the site of injury to promote neuroprotection and axon regeneration.

Embodiment 16. The method of any of embodiments 4-15, wherein the release of CNTF from the hydrogel in the site of injury promotes axon regeneration by prolonging STAT3 signaling.

### REFERENCES

1. D. Kacy Cullen, M. D. Tang-Schomer, L. A. Struzyna, A. R. Patel, V. E. Johnson, J. A. Wolf, D. H. Smith, Microtissue engineered constructs with living axons for targeted nervous system reconstruction. Tissue Eng.-Part A 18, 2280-2289 (2012).
2. B. K. Mueller, R. Mueller, H. Schoemaker, Stimulating neuroregeneration as a therapeutic drug approach for traumatic brain injury. Br. J. Pharmacol. 157, 675-685 (2009).
3. R. Hussain, H. Zubair, S. Pursell, M. Shahab, Neurodegenerative diseases: Regenerative mechanisms and novel therapeutic approaches. Brain Sci. 8 (2018).
4. M. A. Anderson, J. E. Burda, Y. Ren, Y. Ao, T. M. O'Shea, R. Kawaguchi, G. Coppola, B. S. Khakh, T. J. Deming, M. V. Sofroniew, Astrocyte scar formation AIDS central nervous system axon regeneration. Nature 532, 195-200 (2016).
5. J. W. Fawcett, The Struggle to Make CNS Axons Regenerate: Why Has It Been so Difficult? Neurochem. Res. 45, 144-158 (2020).
6. S. G. Varadarajan, J. L. Hunyara, N. R. Hamilton, A. L. Kolodkin, A. D. Huberman. Central nervous system regeneration. Cell. 185, 77-94 (2022).
7. F. T. Afshari, S. K appagantula, J. W. Fawcett, Extrinsic and intrinsic factors controlling axonal regeneration after spinal cord injury. Expert Rev. Mol. Med. 11, 1-19 (2009).
8. F. Sun, K. K. Park, S. Belin, D. Wang, T. Lu, G. Chen, K. Zhang, C. Y eung, G. Feng, B. A. Yankner, Z. He, Sustained axon regeneration induced by co-deletion of PTEN and SOCS3. Nature 480, 372-375 (2011).
9. Z. Álvarez, A. N. Kolberg-Edelbrock, I. R. Sasselli, J. A. Ortega, R. Qiu, Z. Syrgiannis, P. A. Mirau, F. Chen, S. M. Chin, S. Weigand, E. Kiskinis, S. I. Stupp, Bioactive scaffolds with enhanced supramolecular motion promote recovery from spinal cord injury. Science. 374, 848-856 (2021).
10. M. A. Anderson, T. M. O'Shea, J. E. Burda, Y. Ao, S. L. Barlatey, A. M. Bernstein, J. H. Kim, N. D. James, A. Rogers, B. Kato, A. L. Wollenberg, R. Kawaguchi, G. Coppola, C. Wang, T. J. Deming, Z. He, G. Courtine, M. V. Sofroniew, Required growth facilitators propel axon regeneration across complete spinal cord injury. Nature 561, 396-400 (2018).
11. X. Liu, F. Hao, P. Hao, J. Zhang, L. Wang, S. W. You, N. Wang, Z. Y ang, K. F. So, X. Li, Regeneration and functional recovery of the completely transected optic nerve in adult rats by CNTF-chitosan. Signal Transduct. Target. Ther. 8 (2023).
12. P. H. Özdinler, J. D. Macklis, IGF-I specifically enhances axon outgrowth of corticospinal motor neurons. Nat. Neurosci. 9, 1371-1381 (2006).
13. A. H. Dyer, C. Vahdatpour, A. Sanfeliu, D. Tropea, The role of Insulin-Like Growth Factor 1 (IGF-1) in brain development, maturation and neuroplasticity. Neuroscience 325, 89-99 (2016).
14. J. Bloch, E. G. Fine, N. Bouche, A. D. Zurn, P. Aebischer, Nerve growth factor- and neurotrophin-3-releasing guidance channels promote regeneration of the transected rat dorsal root. Exp. Neurol. 172, 425-432 (2001).
15. Z. Yang, H. Duan, L. Mo, H. Qiao, X. Li, The effect of the dosage of NT-3/chitosan carriers on the proliferation and differentiation of neural stem cells. Biomaterials 31, 4846-4854 (2010).
16. Q. Ling, J. A. Herstine, A. Bradbury, S. J. Gray, AAV-based in vivo gene therapy for neurological disorders. Nat. Rev. Drug Discov. 22, 789-806 (2023).
17. E. A. Chowdhury, G. Meno-Tetang, H. Y. Chang, S. Wu, H. W. Huang, T. Jamier, J. Chandran, D. K. Shah, Current progress and limitations of AAV mediated delivery of protein therapeutic genes and the importance of developing quantitative pharmacokinetic/pharmacodynamic (PK/PD) models. Adv. Drug Deliv. Rev. 170, 214-237 (2021).
18. B. Jiang, X. Liu, C. Yang, Z. Yang, J. Luo, S. Kou, K. Liu, F. Sun, Injectable, photoresponsive hydrogels for delivering neuroprotective proteins enabled by metal-directed protein assembly. Sci. Adv. 6, eabc4824 (2020).
19. S. Jin, H. Choi, D. Seong, C. L. You, J. S. Kang, S. Rho, W. B. Lee, D. Son, M. Shin, Injectable tissue prosthesis for instantaneous closed-loop rehabilitation. Nature 623, 58-65 (2023).
20. Z. Wang, Y. Zhang, Y. Yin, J. Liu, P. Li, Y. Zhao, D. Bai, H. Zhao, X. Han, Q. Chen, High-Strength and Injectable Supramolecular Hydrogel Self-Assembled by Monomeric Nucleoside for Tooth-Extraction Wound Healing. Adv. M ater. 34, 1-20 (2022).
21. D. Seliktar, Designing Cell-Compatible Hydrogels for Biomedical Applications. Science. 336, 1124-1128 (2012).
22. J. J. Green, J. H. Elisseeff, Mimicking biological functionality with polymers for biomedical applications. Nature 540, 386-394 (2016).
23. G. Y. Xu, S. Xu, Y. X. Zhang, Z. Y. Yu, F. Zou, X. S. Ma, X. L. Xia, W. J. Zhang, J. Y. Jiang, J. Song, Cell-Free Extracts from Human Fat Tissue with a Hyaluronan-Based Hydrogel Attenuate Inflammation in a Spinal Cord Injury Model through M 2 Microglia/Microphage Polarization. Small 18, 1-14 (2022).
24. F. Feng, X. Song, Z. Tan, Y. Tu, L. Xiao, P. Xie, Y. Ma, X. Sun, J. Ma, L. Rong, L. He, Cooperative assembly of a designer peptide and silk fibroin into hybrid nanofiber gels for neural regeneration after spinal cord injury. Sci. Adv. 9, eadg0234 (2023).
25. S. H. Kim, H. Hong, O. Ajiteru, M. T. Sultan, Y. J. Lee, J. S. Lee, O. J. Lee, H. Lee, H. S. Park, K. Y. Choi, J. S. Lee, H. W. Ju, I.-S. Hong, C. H. Park, 3D bioprinted silk fibroin hydrogels for tissue engineering. Nat. Protoc. 16, 5484-5532 (2021).
26. Z. Zhu, S. Ling, J. Y eo, S. Zhao, L. Tozzi, M. J. Buehler, F. Omenetto, C. Li, D. L. Kaplan, High-Strength, Durable All-Silk Fibroin Hydrogels with Versatile Processability toward Multifunctional Applications. Adv. Funct. M ater. 28, 1-10 (2018).
27. L. Semmler, A. Naghilou, F. Millesi, S. Wolf, A. Mann, S. Stadlmayr, S. Mero, L. Ploszczanski, L. Greutter, A. Woehrer, E. Placheta-Györi, F. Vollrath, T. Weiss, C. Radtke, Silk-in-Silk Nerve Guidance Conduits Enhance Regeneration in a Rat Sciatic Nerve Injury Model. Adv. Healthc. M ater. 12, 1-12 (2023).
28. T. Arndt, U. Chatterjee, O. Shilkova, J. Francis, J. Lundkvist, D. Johansson, B. Schmuck, G. Greco, Å. E. Nordberg, Y. Li, L. U. Wahlberg, M. Langton, J. Johansson, C. Götherström, A. Rising, Tuneable Recombinant Spider Silk Protein Hydrogels for Drug Release and 3D Cell Culture. Adv. Funct. M ater., doi: 10.1002/adfm.202303622 (2023).
29. K. Schacht, T. Scheibel, Controlled hydrogel formation of a recombinant spider silk protein. Biomacromolecules 12, 2488-2495 (2011).
30. K. Schacht, T. Jüngst, M. Schweinlin, A. Ewald, J. Groll, T. Scheibel, Biofabrication of cell-loaded 3D spider silk constructs. Angew. Chemie-Int. Ed. 54, 2816-2820 (2015).
31. W. W. Song, Z. G. Qian, H. Liu, H. F. Chen, D. L. Kaplan, X. X. Xia, On-Demand Regulation of Dual Thermosensitive Protein Hydrogels. A CS Macro Lett. 10, 395-400 (2021).
32. A. Leal-Egaña, T. Scheibel, Silk-based materials for biomedical applications. Biotechnol. Appl. Biochem. 55, 155-167 (2010).
33. X. Wang, C. Yang, X. Wang, J. Miao, W. Chen, Y. Zhou, Y. Xu, Y. An, A. Cheng, W. Y e, M. Chen, D. Song, X. Yuan, J. Wang, P. Qian, A. R. Wu, Z. Y. Zhang, K. Liu, Driving axon regeneration by orchestrating neuronal and non-neuronal innate immune responses via the IFNγ-CGAS-STING axis. Neuron 111, 236-255.e7 (2023).
34. M. A. Calhoun, S. A. Bentil, E. Elliott, J. J. Otero, J. O. Winter, R. B. Dupaix, Beyond Linear Elastic Modulus: Viscoelastic Models for Brain and Brain Mimetic Hydrogels. ACS Biomater. Sci. Eng. 5, 3964-3973 (2019).
35. M. Iwashita, N. Kataoka, K. Toida, Y. Kosodo, Systematic profiling of spatiotemporal tissue and cellular stiffness in the developing brain. Dev. 141, 3793-3798 (2014).
36. K. Liu, A. Tedeschi, K. K. Park, Z. He, Neuronal intrinsic mechanisms of axon regeneration. Annu. Rev. Neurosci. 34, 131-152 (2011).
37. V. Pernet, S. Joly, D. Dalkara, N. Jordi, O. Schwarz, F. Christ, D. V. Schaffer, J. G. Flannery, M. E. Schwab, Long-distance axonal regeneration induced by CNTF gene transfer is impaired by axonal misguidance in the injured adult optic nerve. Neurobiol. Dis. 51, 202-213 (2013).
38. Z. He, Y. Jin, Intrinsic Control of Axon Regeneration. Neuron 90, 437-451 (2016).
39. C. Yang, X. Wang, J. Wang, X. Wang, W. Chen, N. Lu, S. Siniossoglou, Z. Y ao, K. Liu, Rewiring Neuronal Glycerolipid Metabolism Determines the Extent of Axon Regeneration. Neuron 105, 276-292.e5 (2020).

We claim:

1. An injectable protein delivery system, comprising recombinant spider silk protein spidroin-SpyTag, wherein the spidroin-SpyTag is in injectable hydrogel form, and wherein the spidroin-SpyTag is covalently conjugated to one or more bioactive agents.

2. The system of claim 1, wherein the one or more bioactive agents comprise one or more protein therapeutics.

3. The system of claim 2, wherein the one or more protein therapeutics comprise one or more of ciliary neurotrophic factor (CNTF), insulin-like growth factor (IGF1), laminin, or osteopontin (OPN).

4. A method for delivering a therapeutic agent to a central nervous system (CNS) targeted tissue, the method comprising administering an effective amount of the injectable protein delivery system of claim 1 to a targeted CNS tissue of a subject.

5. The method of claim 4, wherein the subject is a mammal.

6. The method of claim 5, wherein the mammal is a mouse or human.

7. The method of claim 4, wherein the spidroin-SpyTag transitions from sol to gel state after brief sonication and incubation at a temperature of about 37° C. for injectability.

8. The method of claim 7, wherein the spidroin-SpyTag is covalently conjugated with one or more protein therapeutics.

9. The method of claim 8, wherein the one or more protein therapeutics comprise one or more of ciliary neurotrophic factor (CNTF), insulin-like growth factor (IGF1), laminin, or osteopontin (OPN).

10. The method of claim 4, wherein an effective amount of the hydrogel comprising the spidroin-SpyTag is covalently conjugated with one or more protein therapeutics and is administered to the subject via intravitreous, intrathecal, intramuscular, intradermal, intracranial, intraspinal, or epidural injection.

11. The method of claim 10, wherein the subject is affected by a CN S disorder or injury.

12. The method of claim 11, wherein the CNS disorder or injury comprises spinal cord injury, traumatic brain injury, stroke, glaucoma, muscle dystrophy, muscle hypertrophy, metabolic myopathies, or muscle paralysis.

13. The method of claim 12, wherein a site of injury comprises an optic nerve and a retinal tissue.

14. The method of claim 12, wherein the hydrogel is injected in a CNS site of injury to promote neuroprotection and axon regeneration.

15. The method of claim 13, wherein the hydrogel is injected in the site of injury to promote neuroprotection and axon regeneration.

16. The method of claim 12, wherein the release of CNTF from the hydrogel in the site of injury promotes axon regeneration by prolonging STAT3 signaling.

17. The method of claim 13, wherein the release of CNTF from the hydrogel in the site of injury promotes axon regeneration by prolonging STAT3 signaling.


SEQUENCE LISTING (8 sequences)

SEQ ID NO: 1 - DNA, 39 bp, synthetic construct
  source 1..39: mol_type = other DNA
gcacatattgttatggttgacgcttataagccaacaaaa

SEQ ID NO: 2 - DNA, 348 bp, Streptococcus pyogenes
  source 1..348: mol_type = genomic DNA
gccatggttgataccttatcaggtttatcaagtgagcaaggtcagtccggtgatatgacaattgaagaagatagtgctacccatattaaattctcaaaacgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgacgctcatattgac

SEQ ID NO: 3 - protein, 13 aa, synthetic construct
AHIVMVDAYKPTK

SEQ ID NO: 4 - protein, 379 aa, synthetic construct
HMSHTTPWTNPGLAENFMNSFMQGLSSMPGFTASQLDDMSTIAQSMVQSIQSLAAQGRTSPNKLQALNMAFASSMAEIAASEEGGGSLSTKTSSIASAMSNAFLQTTGVVNQPFINEITQLVSMFAQAGMNDVSAGNSGRGQGGYGQGSGGNAAAAAAAAAAAAAAAGQGGQGGYGRQSQGAGSAAAAAAAAAAAAAAGSGQGGYGGQGQGGYGQSGNSVTSGGYGYGTSAAAGAGVAAGSYAGAVNRLSSAEAASRVSSNIAAIASGGASALPSVISNIYSGVVASGVSSNEALIQALLELLSALVHVLSSASIGNVSSVGVDSTLNVVQDSVGQYVGGTGGGGSGGGGSGGGGSASAHIVMVDAYKPTKLEHHHHHH

SEQ ID NO: 5 - protein, 488 aa, synthetic construct
MKGSSHHHHHHVDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIDGPQGIWGQLEGHGVGVPGVGVPGVGVPGEGVPGVGVPGVGVPGVGVPGVGVPGEGVPGVGVPGVGVPGVGVPGVGVPGEGVPGVGVPGVGELTFALRGDNPVPMSMRGGKLTWQELYQLKYKGITSVPGVGVPGVGVPGEGVPGVGVPGVGVPGVGVPGVGVPGEGVPGVGVPGVGVPGVGVPGVGVPGEGVPGVGVPGVGVPGGLVDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIDGPQGIWGQLEWKK

SEQ ID NO: 6 - protein, 655 aa, synthetic construct
MKGSSHHHHHHVDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIDGPQGIWGQLEGHGVGVPGVGVPGVGVPGEGVPGVGVPGVGVPGVGVPGVGVPGEGVPGVGVPGVGVPGVGVPGVGVPGEGVPGVGVPGVGELAFAEQTPLTLHRRDLCSRSIWLARKIRSDLTALMESYVKHQGLNKNINLDSVDGVPVASTDRWSEMTEAERLQENLQAYRTFQGMLTKLLEDQRVHFTPTEGDFHQAIHTLMLQVSAFAYQLEELMVLLEQKIPENEADGMPATVGDGGLFEKKLWGLKVLQELSQWTVRSIHDLRVISSHQMGISALESHYGAKDKQMTSVPGVGVPGVGVPGEGVPGVGVPGVGVPGVGVPGVGVPGEGVPGVGVPGVGVPGVGVPGVGVPGEGVPGVGVPGVGVPGGLVDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIDGPQGIWGQLEWKK

SEQ ID NO: 7 - protein, 526 aa, synthetic construct
MKGSSHHHHHHVDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIDGPQGIWGQLEGHGVGVPGVGVPGVGVPGEGVPGVGVPGVGVPGVGVPGVGVPGEGVPGVGVPGVGVPGVGVPGVGVPGEGVPGVGVPGVGELGPETLCGAELVDALQFVCGDRGFYFNKPTGYGSSSRRAPQTGIVDECCFRSCDLRRLEMYCAPLKPAKSATSVPGVGVPGVGVPGEGVPGVGVPGVGVPGVGVPGVGVPGEGVPGVGVPGVGVPGVGVPGVGVPGEGVPGVGVPGVGVPGGLVDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIDGPQGIWGQLEWKK

SEQ ID NO: 8 - protein, 754 aa, synthetic construct
MKGSSHHHHHHVDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIDGPQGIWGQLEGHGVGVPGVGVPGVGVPGEGVPGVGVPGVGVPGVGVPGVGVPGEGVPGVGVPGVGVPGVGVPGVGVPGEGVPGVGVPGVGELIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVNTSVPGVGVPGVGVPGEGVPGVGVPGVGVPGVGVPGVGVPGEGVPGVGVPGVGVPGVGVPGVGVPGEGVPGVGVPGVGVPGGLVDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHIDGPQGIWGQLEWKK